(19) United States
(12) Patent Application Publication
(10) Pub. No.: US 2017/0143646 A1
(43) Pub. Date: May 25, 2017

(54) EFFECT OF LIPOPHILIC NUTRIENTS ON DIABETIC EYE DISEASES

(71) Applicant: OmniActive Health Technologies Limited, Mumbai (IN)

(72) Inventors: Jayant DESHPANDE, Charlottetown (CA); Shankaranarayanan JEYAKODI, Thane (W) (IN)

(21) Appl. No.: 15/300,210

(22) PCT Filed: Mar. 26, 2015
(86) PCT No.: PCT/IB2015/052248

§ 371 Date: Sep. 28, 2016

(30) Foreign Application Priority Data
Mar. 28, 2014 (IN) 1128/MUM/2014

(51) Int. Cl. A61K 31/12; A61K 9/00; A23L 33/105; A61K 31/047

(52) U.S. Cl. CPC A61K 31/12; A61K 31/047; A61K 9/0056; A23L 33/105; A23V 2002/00

(57) ABSTRACT

Compositions containing molecular dispersions of lipophilic nutrients and methods thereof are provided for delaying the development and maturation of eye related complications of diabetes by administering a composition containing lipophilic nutrients. More particularly, methods relate to delaying the development and maturation of eye related complications of diabetes by administering a composition containing lutein and its isomers, lutein ester, zeaxanthin isomers, turmeric extract, curcumin or curcuminoids, derived from plant extract/oleoresin containing xanthophylls/xanthophylls esters which are safe for human consumption and are particularly useful as dietary supplements for nutrition and health promoting benefits.

### FIELD

The present invention relates to a method of delaying the development and maturation of eye related complications of diabetes by administering a composition containing lipophilic nutrients. More particularly, the present invention relates to a method of delaying the development and maturation of eye related complications of diabetes by administering a composition containing lutein and its isomers, lutein ester, zeaxanthin isomers, turmeric extract, curcumin or curcuminoids, either alone or in combination thereof, derived from plant extract/oleoresin containing xanthophylls/xanthophylls esters which are safe for human consumption and are particularly useful as dietary supplements for nutrition and health promoting benefits.

### BACKGROUND OF THE INVENTION

Eye is the most important and complex organ in the human body that is protected by the bony orbit of the eye. It is divided into anterior segment which consists of the cornea, iris, lens, ciliary body, and the anterior portion of the sclera. The posterior segment is bounded anteriorly by the lens and extends to the back of the eye. The retina optic disc is also included in the posterior segment. The light passes through anterior portion called cornea, aqueous humor, crystalline lens, pupil, vitreous humor to reach the retina of the eye, this pathway is called visual axis of the eye. The lens refracts light rays and helps focus the image of an object on the retina (fovea) through accommodation.

Diabetes and diabetic complications: Diabetes is one of the most occurring non-communicable, heterogeneous, metabolic disorder characterized by hyperglycemia resulting from defective insulin production, resistance to insulin action or both, there are two forms of diabetes, type 1 and type 2. Type 1 diabetes mellitus is the consequence of an autoimmune-mediated destruction of pancreatic β-cells, leading to insulin deficiency.

Type 2 diabetes mellitus is characterized by insulin resistance and relative, rather than absolute, insulin deficiency. According to the latest World Health Organization (WHO) estimation currently there are about 366 million diabetic people in the world, it is expected to increase to 552 million by 2030 and India has about 62 million diabetics. Prolonged exposure to chronic hyperglycemia can lead to various complications including vascular and non-vascular complications. Vascular complications are further divided into macrovascular and microvascular complications. Tissues like retina, kidney, peripheral nerves and lens are most affected by long term complications of the diabetes, which results in the development of diabetic retinopathy, nephropathy, neuropathy and cataract respectively.

Diabetic cataract: Cataract is characterized by opacity of the eye lens, and the leading cause of blindness worldwide. The development of cataract in diabetics is 2-5 times more when compared with the non-diabetic counterparts. Furthermore, patients with diabetes mellitus have higher complication rates from cataract surgery. Both diabetes and cataract pose an enormous health and economic burden, particularly in developing countries, where diabetes treatment is insufficient and cataract surgery often inaccessible. Many clinical interventions have been reported to countering cataract including diabetic cataract but are not completely successful at clinical practice.

Diabetic Retinopathy: Diabetic retinopathy (DR) is one of the most common micro vascular complications of diabetes. DR occurs in 70% of all persons having diabetes for more than 15 years and is the most common cause of blindness. DR is a disease of retina, resulting in loss of vision, macular edema, recurrent vitreous hemorrhages, tractional or secondary rhegmatogenous retinal detachment, and so forth. Since the last two decades there have been significant developments in the emerging field of pharmacotherapy of DR. The advent of laser photocoagulation three decades back, was really useful in limiting vision loss in most of the cases and is still considered the gold standard therapy for the treatment of DR. However, corticosteroids and anti-VEGF agents have shown promising results with regard to prevention of neo-vascularisation, but remained limited in use due to their short-duration effects. Therefore, pharmacotherapy of DR is still an adjunct to pan retinal photocoagulation.

In recent years, a great deal of attention has been focused on biological activities of carotenoids. Carotenoids are naturally occurring xanthophylls in plants that are involved in light harvesting reactions and protection of plant organelles against singlet oxygen induced damage. Dietary carotenoids serve as antioxidants in the tissues (Thurnham D L. Carotenoids: function and fallacies. Proc Nutr Soc 1994; 53: 77-87) and protect the body from oxidative damage.

Mammalian species do not synthesize carotenoids and therefore these have to be obtained from dietary sources such as fruits and vegetables and/or dietary supplements. Numerous epidemiological studies support a strong inverse relationship between consumption of carotenoid rich fruits and vegetables and incidence of degenerative diseases (Coleman H, Chew E. Nutritional supplementation in age-related macular degeneration. Curr Opin Ophthalmol 2007; 18(3): 220-223)

Lutein is one of the major xanthophylls present in green leafy vegetables and egg yolk. Lutein and zeaxanthin are known to selectively accumulate in the macula of the human retina. They have been thought to work as antioxidants and as blue light filters to protect the eyes from such oxidative stresses as cigarette smoking and sunlight exposure, which can lead to age-related macular degeneration and cataracts.

Xanthophylls can show both optical (R- and S-stereo isomers) and geometrical isomers (trans, E- and cis, Z-). The conformation of R- and S-stereo isomers is based on circular dichroism (CD) spectral and chiral column high-performance liquid chromatography (HPLC) studies while the conformation of cis- and trans-isomers is based on electronic, infrared, nuclear magnetic resonance (NMR), high-performance liquid chromatography-mass spectrometry (HPLC-MS) and high-performance liquid chromatography-nuclear magnetic resonance (HPLC-NMR) on-line spectroscopy studies. It is well known that when an organic molecule has a carbon atom with four different types of atoms or groups attached to it, that carbon atom is designated as chiral carbon atom. The chiral carbon atom is responsible for two different spatial arrangements leading to the formation of optical isomers while the number of double bonds of the polyene chain and the presence of a methyl group and the absence of steric hindrance decide the number of trans- and cis-isomers. In the case of trans-zeaxanthin, the carbon atoms at 3 and 3′ positions in the two end rings are both chiral atoms.

Thus, trans-zeaxanthin has two chiral centers at the carbon atoms C3 and C3′, based on the positions of the secondary hydroxy groups attached to them. Therefore, there are four possible stereo isomers of trans-zeaxanthin namely, (3R-3′R)-isomer, (3S-3′S)-isomer and (3R-3′S)- or (3S-3′R)-isomer. In these isomers (3R-3′S)- & (3S-3′R)- are identical. Thus, there are three chiral isomers of trans-zeaxanthin. The isomer causing rotation of polarized light in a right handed manner is called R-stereo isomer, the isomer causing left handed rotation is called S-stereo isomer, and the third isomer possessing twofold opposite effects (R,S; optically inactive) which is called meso-form of zeaxanthin.

The conjugated double bonds of lutein and zeaxanthin contribute to the distinctive colors of each pigment, and also influence the ability of these to quench singlet oxygen. Due to the extra conjugated double bond, zeaxanthin is believed to be a stronger anti-oxidant compared to lutein.

Regarding the location of xanthophylls at a cellular level, they are reported to be bound to specific proteins referred to as xanthophylls binding protein (XBP). The XBP is suggested to be involved in the uptake of lutein and zeaxanthin from the blood stream and stabilization of the same in the retina. The study of xanthophylls and XBP by femto-second transient absorption spectroscopy showed better stability for (3R,3′S)-zeaxanthin enriched XBP compared to (3R,3′R)-zeaxanthin while the photo physical properties of the xanthophylls: (3R,3′R)-zeaxanthin and (3R, 3′S,meso)-zeaxanthin are generally identical. It is likely that the meso-zeaxanthin is better accommodated with XBP wherein the protein protects the xanthophylls from degradation by free radicals. Thus, the complex may be a better antioxidant than the free xanthophylls, facilitating improved protection of ocular tissue from oxidative damage. (Billsten et al., Photophysical Properties of Xanthophylls in Caroteno proteins from Human Retina, Photochemistry and Photobiology, 78, 138-145, 2003)

Epidemiological studies suggested that higher dietary intake of lutein and zeaxanthin reduces the risk of cataracts and age-related macular degeneration. Previous studies showed that rats treated with combination of insulin and lutein exhibited delayed development and maturation of cataract than treated with lutein or insulin alone. Serum lutein and zeaxanthin concentrations in DR patients were found to be significantly lower than those in normal subjects, and their intake was proved to improve the visual acuity, contrast sensitivity, and macular edema, suggesting that lutein and zeaxanthin supplementation might be targeted as potential therapeutic agents in treating DR.

Curcumin has been identified as the active principle of turmeric and has been shown to exhibit antioxidant, anti-inflammatory, antimicrobial, and anticarcinogenic activities. Curcumin is a natural extract from the spice Turmeric. Turmeric is derived from the plant Curcuma Longa, a member of the ginger family. Curcumin is a known antioxidant which inherently has many health benefits. Curcumin was shown to induce apoptosis in human retinal endothelial cells and decrease VEGF release into media in vitro and it also inhibits diabetes-induced elevation of serum VEGF levels in rat. Vascular endothelial growth factor (VEGF) expression, induced by high glucose levels and hypoxia, is a main feature in retinopathy. Several studies have also shown that VEGF may also play a role in the development of the earliest stages of retinopathy.

Though dietary supplements such as curcumin, lutein, zeaxanthin, etc have offered some benefits in preclinical studies, the translation has been very poor and the doses used in clinical trials are unfeasible to practice in reality. One of the major reasons for the lack of clinical success with curcumin is linked to its extensive intestinal and hepatic metabolic biotransformation resulting in poor bioavailability. Recently, the focus is to address bioavailability concerns of the supplements with a view to improve the therapeutic efficacy.

The lipophilic nutrients such as curcumin, lutein, zeaxanthin, ginger, etc are poorly absorbed if administered either as oil suspensions or as beadlets, which are the currently used forms. The main reason for poor absorption is their poor solubility in water. Due to their insolubility their bioavailability is very poor. Lipophilic nutrients have limited absorption in the body due to limited solubility in the gastrointestinal tract. Generally, the bioavailability of such nutrients is below 40%. The bioavailability can be enhanced by reducing the particle size, which in turn will enhance their efficiency of micellization. Dispersion of nutritional products at molecular level is generally regarded as a technique of reducing the particle size. Such molecular dispersions provide higher efficiency for micellization of nutrients in water and thereby increase the bioavailability.

Hence, it is interesting to search the effects of a composition containing soluble lipophilic nutrients in diabetic rats with respect to its beneficial effect on retina by a nutrigenomics approach and the effect was compared with regular lipophilic nutrients.

Nutrigenomics is the science of interaction between nutrients and genes. It is the study of how genetic expression affect your need for certain nutrients and help maintain optimal health throughout your life. Nutrigenomics promotes an increased understanding of how nutrition influences metabolic pathways and homeostatic control, how this regulation is disturbed in the early phases of diet-related diseases, and the extent to which individual sensitizing genotypes contribute to such diseases. Our goal is to better understand how phytonutrients affect gene expression.

Numerous references are available that provide compositions containing carotenoids used for the prevention/treatment of diabetic eye diseases.

In Brown et al. (Am J Clin Nutr. 1999), dietary antioxidants, including carotenoids, are hypothesized to decrease the risk of age-related cataracts by preventing oxidation of proteins or lipids within the lens. However, prospective epidemiologic data concerning this phenomenon are limited. The authors examined prospectively the association between carotenoid and vitamin A intake and cataract extraction in men. US male health professionals (n=36644) who were 45-75 y of age in 1986 were included in this prospective cohort study. Others were subsequently included as they became 45 y of age. During 8 y of follow-up, 840 cases of senile cataract extraction were documented. They observed a modestly lower risk of cataract extraction in men with higher intakes of lutein and zeaxanthin but not of other carotenoids (alpha-carotene, beta-carotene, lycopene, and beta-cryptoxanthin) or vitamin A after other potential risk factors, including age and smoking, were controlled for. Men in the highest fifth of lutein and zeaxanthin intake had a 19% lower risk of cataract relative to men in the lowest fifth (relative risk: 0.81; 95% CI: 0.65, 1.01; P for trend=0.03). Among specific foods high in carotenoids, broccoli and spinach were most consistently associated with a lower risk of cataract. Lutein and zeaxanthin may decrease the risk of cataracts severe enough to require extraction, although this relation appears modest in magnitude. This study is a cohort study done with the US male population. This study establishes a relation between nutrition deficiency and the occurrence of cataract.

EP 2618832 A2 relates to a composition comprising an enzyme selected from the group comprising superoxide dismutase (SOD) and SOD mimics and the like, in association with lutein and at least one stereoisomer of zeaxanthin; and also includes a kit of parts comprising such composition, wherein the kit comprises a first part comprising the enzyme, and a second part comprising lutein and at least one zeaxanthin isomer. The composition or the kit of parts may be included in a functional food, a nutraceutical composition or a food or dietary supplement, a medicament or a pharmaceutical composition, or a veterinarian product. The reference also relates to a composition for use in treating, preventing, and/or stabilizing a disease, condition and/or disorder of the eye associated with oxidative stress, by administering to a subject in need thereof a medicament or a pharmaceutical composition. However, the reference does not make use of zeaxanthin isomers.

WO2010032267A2 relates to an herbal formulation for prevention and treatment of diabetes and associated complications comprising extracts from selected Indian medicinal herbs. The reference relates to associated formulations for different diabetes related complications, which are individually useful in clinical requirements, such as improving renal health, preventing renal diseases, preventing diabetic retinopathy, and/or in the prevention and treatment of oxidative damage to the heart and/or blood vessels. The formulations are versatile and can be processed into extracts/concentrates and further pharmacologically modified into tablets or capsules or granules or syrups or herbal health drinks or inhalable herbal medicinal preparations or ocular preparations or transdermal absorbable preparations such as ointments/gels or injectable medicine. This is a poly herbal formulation and there is no synergistic data to support the claim.

CN 102178925A relates to a lutein ophthalmic preparation for protecting eyesight, which is prepared from the following raw materials: 5 to 13 parts of water-soluble lutein (based on C4oH56O2), 50 to 80 parts of taurine, 0.1 to 0.5 parts of selenium (based on Se), 10 to 25 parts of zinc (based on Zn), 0.5 to 1.0 part of water-soluble vitamin A, and 0.8 to 2.0 parts of glutathione; an auxiliary material consists of a diluent, a wetting agent, an isoosmotic adjusting agent, a preservative, an antioxidant and water for injection; formulations comprise eye drops, eye lotion and the like; and the lutein ophthalmic preparation is suitable for eye diseases such as myopia, long sightedness, cataract, glaucoma, retinal pigment degeneration, macular degeneration and the like. Various nutrition factors are reasonably compatible, a blank of a lutein external preparation is filled, and the bioavailability and health-care effect are obviously improved; and through actual application by 300,000 people, the total effective rate for the myopia, cataract and diabetic eye disease is over 90 percent and the lutein ophthalmic preparation has positive promotion value. This ophthalmic preparation contains only one macular carotenoid i.e. lutein and does not mention use of zeaxanthin isomers.

In Sasaki et al. (IOVS, March 2009, Vol. 50, No. 3), the aim of this study was to investigate, with the use of a mouse endotoxin-induced uveitis (EIU) model, the neuroprotective effects of lutein against retinal neural damage caused by inflammation. EIU was induced by intraperitoneal injection of lipopolysaccharide (LPS). Each animal was given a subcutaneous injection of lutein or vehicle three times: concurrently with and 3 hours before and after the LPS injection. Analysis was carried out 24 hours after EIU induction. Levels of rhodopsin protein and signal transducer and activator of transcription 3 (STAT3) activation were analysed by immunoblotting. Lengths of the outer segments of the photoreceptor cells were measured. Dark-adapted full-field electroretinograms were recorded. Oxidative stress in the retina was analyzed by dihydroethidium and fluorescent probe. Expression of glial fibrillary acidic protein (GFAP) was shown immunohistochemically. The EIU-induced decrease in rhodopsin expression followed by shortening of the outer segments and reduction in a-wave amplitude were prevented by lutein treatment. Levels of STAT3 activation, downstream of inflammatory cytokine signals, and reactive oxygen species (ROS), which are both upregulated during EIU, were reduced by lutein.

Pathologic change of Muller glial cells, represented by GFAP expression, was also prevented by lutein. The present data revealed that the antioxidant lutein was neuroprotective during EIU, suggesting a potential approach for suppressing retinal neural damage during inflammation. Lutein is a nutritional supplement and subjects have to take daily dose for prevention or treatment of any disease. In this study, Lutein was administered through injection. Daily supplementation of lutein via injection is painful causing discomfort to the subject.

CA 2760932 A1 relates to ophthalmic formulations that deliver a variety of therapeutic agents, including but not limited to rapamycin (sirolimus), analogs thereof (rapalogs) or other mammalian target of rapamycin (mTOR) inhibitors, to a subject for an extended period of time. The ophthalmic formulations may be placed in an aqueous medium of a subject, including but not limited to intraocular or periocular administration, or placement proximate to a site of a disease or condition to be treated in a subject. A method may be used to administer a therapeutic agent to treat or prevent age-related macular degeneration, macular edema, diabetic retinopathy, uveitis, dry eye, or a hyperpermeability disease in a subject.

### SUMMARY

Overcoming the difficulty of delivering therapeutic/preventive agents to specific regions of the eye presents a major challenge to treatment of most eye disorders. Due to poor bioavailability of lipophilic nutrients the delivery of many potentially important therapeutic/preventive agents to the eye is hindered.

From above it is clear that there is a need to provide a technology which can overcome the difficulty of delivering the therapeutic/preventive agents for diabetic eye complications even at reduced dose levels.

Molecular dispersions of lipophilic nutrients are provided, which are useful for delaying the development and maturation of eye related complications of diabetes and which are safe for human consumption and are particularly useful as dietary supplements for nutrition and health promoting benefits.

In one embodiment, molecular dispersions of lipophilic nutrients such as curcumin or trans-lutein and zeaxanthin isomers namely (R,R)-zeaxanthin and (R,S)-zeaxanthin or trans-lutein and (R,R)-zeaxanthin in a solid or liquid hydrophilic carrier, derived from plant extract/oleoresin containing xanthophylls/xanthophylls esters are provided, and which are useful for delaying the development and maturation of eye related complications of diabetes.

In one embodiment, molecular dispersions of a composition are provided, which contain at least 80% by weight of total xanthophylls, out of which the trans-lutein content is 80-95% w/w; (R,R)-zeaxanthin is 14-20% w/w; (R,S)-zeaxanthin is 0.01-1% w/w or trans-lutein content is 80-95% w/w; (R,R)-zeaxanthin is 14-20% w/w, and traces of other carotenoids derived from the plant extracts/oleoresin containing xanthophylls/xanthophylls esters or curcumin which contains 5-95% of curcuminoids.

In one embodiment, molecular dispersions of a xanthophyll composition containing trans-lutein and zeaxanthin isomers namely (R,R)-zeaxanthin and (R,S)-zeaxanthin, or trans-lutein and (R,R)-zeaxanthin, in a solid or liquid hydrophilic carrier are provided, wherein the composition has higher antioxidant potential than the free xanthophylls and which are useful for delaying the development and maturation of eye related complications of diabetes.

In one embodiment, molecular dispersions of a curcumin composition containing curcuminoids in a solid or liquid hydrophilic carrier are provided, and which are useful for delaying the development and maturation of eye related complications of diabetes.

In one embodiment, molecular dispersions of lipophilic nutrients are provided, which have higher efficiency for micellization which enhances the bioavailability resulting in increased levels of lipophilic nutrients in tissues, in which these molecular dispersions are effective at relatively lower concentrations and are useful for delaying the development and maturation of eye related complications of diabetes.

In one embodiment, molecular dispersions of lipophilic nutrients in solid or liquid hydrophilic carriers are provided, which have higher bioavailability.

In some embodiments, the molecular dispersions of lipophilic nutrients which are prepared by using safe solvents (GRAS) and are suitable for human consumption, with minimum solvent residues.

Further advantages of the compositions and/or methods herein will become apparent from a consideration of the ensuing description.

The usefulness of the products, compositions, and/or methods described herein below, which are illustrated in the examples, should not be construed to limit the scope of the present innovations in any manner whatsoever.

Methods herein are related to delaying the development and maturation of eye related complications of diabetes by administering a composition containing lipophilic nutrients. More particularly, methods herein are related to delaying the development and maturation of eye related complications of diabetes by administering a composition containing lutein and its isomers, lutein ester, zeaxanthin isomers, turmeric extract, and/or curcumin or curcuminoids, derived from plant extract/oleoresin containing xanthophylls/xanthophylls esters, and which are safe for human consumption and are particularly useful as dietary supplements for nutrition and health promoting benefits.

The molecular dispersions herein are in the form of powders, tablets, capsules, sachets, beadlets, microencapsulated powders, oil suspensions, liquid dispersions, pellets, soft gel capsules, chewable tablets or liquid preparations.

Molecular dispersions herein of trans-lutein and zeaxanthin isomers namely (R,R)-zeaxanthin and (R,S)-zeaxanthin, or of trans-lutein and (R,R)-zeaxanthin, and/or of curcumin containing curcuminoids in a solid or liquid hydrophilic carrier have enhanced water solubility and bioavailability, which helps in effectively delivering the molecules and shows potential in delaying the development and maturation of eye related complications of diabetes. The use of carotenoids namely, trans-lutein and zeaxanthin isomers having higher antioxidative potential in highly water soluble form with enhanced bioavailability for delaying the development and maturation of eye related complications of diabetes has not yet been reported in earlier literature.

In some embodiments, compositions herein are molecular dispersions of hydrophilic liquid and solid carriers. In some embodiments, a process for making compositions herein includes making them into molecular dispersions of hydrophilic liquid and solid carriers, which enhances water solubility of the nutrients which characteristics are beneficial for formulating further as beverage or soft gelatin capsule or as licaps.

Compositions herein in some embodiments include a water soluble, molecular dispersion of lipophilic nutrients comprising:

(a) a stabilizer,

(b) a water soluble hydrophilic carrier, and

(c) and optionally a surfactant,

and which are useful for converting oily nutrients into powders, tablets, capsules, ointments, pastes, lotions, liniments, mouthwashes, sachets, gargles and which are suitable for incorporation into beverages.

In some embodiments, compositions herein are free flowing water soluble molecular dispersions of lipophilic nutrients such as lutein, zeaxanthin, beta carotene and lycopene in water soluble liquid or solid hydrophilic carriers which can be formulated further as beverage or soft gelatin capsule or as licaps.

In some embodiments, a process for the preparation of free flowing water soluble molecular dispersions of lipophilic nutrients such as lutein, zeaxanthin, beta carotene and lycopene in water soluble liquid or solid hydrophilic carriers which can be formulated further as beverage or soft gelatin capsule or as licaps, is provided.

In some embodiments, a solution of lipophilic nutrient in a polar or non polar organic solvent can be dispersed in certain water soluble hydrophilic liquid or solid carrier systems. Upon removal of solvent under vacuum, the resultant dispersion remains as a homogenous liquid or solid dispersion which is suitable for filling in to soft gel capsules or in to licaps. Such liquid or solid dispersions are suitable for filling in to capsules or for making granules, tablets, filling in to sachets or for making beverages.

In some embodiments, compositions herein are free flowing water soluble molecular dispersions of lipophilic nutrients such as lutein, zeaxanthin, beta carotene and lycopene in water soluble hydrophilic liquid or solid carriers, which are useful for converting into soft gelatin capsules, licaps, ointments, pastes, lotions, liniments, mouthwashes, gargles etc. which are also suitable for incorporation in to beverages.

In some embodiments, a process is provided for the preparation of free flowing water soluble molecular dispersions of lipophilic nutrients in water soluble hydrophilic liquid or solid carriers, which are useful for converting into soft gelatin capsules, licaps, ointments, pastes, lotions, liniments, mouthwashes, gargles etc. which are also suitable for incorporation in to beverages which comprises, dissolving a lipophilic nutrient in a non polar/polar solvent or a mixture thereof to form a solution;

(ii) filtering the resulting solution to remove insoluble impurities;

(iii) separately, dissolving the water soluble hydrophilic liquid or solid carrier, stabilizer and optionally, a surfactant in a polar solvent to form a clear solution;

(iv) blending the solution obtained in step (i) with the solution obtained in step (iii);

(v) heating the resulting mixture to remove the solvent at a temperature in the range of 20 -45 deg C. and at a pressure ranging between 500-760 mm of mercury;

(vi) cooling the resultant molecular dispersion to ambient temperature; and

(vii) passing the cooled molecular dispersion obtained in step (vi) through a sieve of appropriate mesh size to remove any agglomerate or lumps to produce a free flowing water soluble or solid dispersion of lipophilic nutrients.

The term ‘lipophilic’ though refers to lipid-like, it generally covers all compounds that are poorly water soluble. Thus, the scope of the term includes poorly water soluble amino acids, proteins, minerals, herbal extracts such as curcumin, carbohydrates, alkaloids, flavonoids and glycosides.

The lipophilic nutrient which can be used includes, but is not limited to lutein, lutein ester, zeaxanthin isomers, lycopene, beta carotene, tocopherols, astaxanthin, omega-3 fatty acids, ubiquinone, phytosterols, lecithins and the mixtures thereof.

The water soluble hydrophilic liquid or solid carrier used for forming the dispersing solution includes polyethylene glycol 200, polyethylene glycol 400, ethylene glycol, propylene glycol, glycerol, sorbitol, glucose syrup, corn steep liquor mannitol, polyethylene glycol 6000, polyethylene glycol 10000, Polyethylene glycol 20000, polyvinyl pyrrolidone, hydroxyl propyl methyl cellulose, sucrose, glucose, sodium chloride, hydroxyl propyl cellulose, polyvinyl alcohol, soluble starch, hydrolyzed starch and mixtures thereof.

In some embodiments the solvent employed for preparing the solution of the lipophilic nutrient product may be selected from Acetone, Hexane, Ethyl Acetate, Isopropyl Alcohol, Ethanol, Dichloromethane, Methanol, etc., more preferably form Acetone, Ethanol, Dichloromethane, Isopropyl alcohol, and more preferably Dichloro Methane and Isopropyl Alcohol.

The stabilizer which may be used in the process may be selected from ascorbic acid, BHA, BHT, ascorbyl palmitate, rosemary extract, mixed natural tocopherols, alpha tocopheryl acetate, sodium ascorbate, castor oil derivatives, sodium lauryl sulfate and mixtures thereof.

The surfactant which may be used in the process may be selected from polysorbate 20, polysorbate 60, polysorbate 80, sodium lauryl sulfate and mixtures thereof.

The heating step in vacuum for evaporating the solvent present in the nutrient dispersion may be carried at a temperature preferably in the range of 35 to 45 deg C.

In some embodiments, the lipophilic nutrient is dispersed in a water soluble hydrophilic liquid or solid carrier, at a molecular level, so that its solubility and consequently, its bioavailability is enhanced several fold. In dispersing hydrophilic nutrients in hydrophilic liquid or solid carriers the resulting dispersions of the nutrients have significantly higher solubility and bioavailability.

Further, dispersing the lipophilic nutrients in a hydrophilic liquid or solid carrier helps in the formulation of lipophilic nutrient in the forms such as hard gelatin capsule and soft gelatin capsule.

For achieving a molecular dispersion, the lipophilic nutrient needs to be dissolved in a polar or non polar solvent. Depending on the chemical nature of the lipophilic nutrient, a polar or a non-polar or a mixture of polar and non-polar solvent can be used. If required, the mixture of the lipophilic nutrient and the solvent may be warmed to enhance the rate of dissolution. Quite often, the lipophilic nutrient requires a stirring for additional period of time to complete the dissolution. To ensure the complete dissolution, it may be necessary to pass the resultant solution through a filter medium and use only the filtrate for molecular dispersion. If the viscosity of the solution is high, it may be further diluted with the solvent used for dissolution, so that filtration step can be performed faster.

The hydrophilic (water soluble) carrier is dissolved in a suitable polar solvent such as ethanol, isopropyl alcohol, acetone, methanol, propylene glycol and/or water to form clear solution. The hydrophilic water soluble carrier used for dispersion may also be mixed with stabilizers and optionally with a surfactant. If required, the stabilizers may be required to be dissolved in solvent before mixing with the hydrophilic carrier. If the resultant mixture is not a clear solution, the solution may be filtered discarding the residue.

The dispersion of the lipophilic nutrient is then mixed with the hydrophilic liquid or solid carrier to obtain a homogenous mass. For this purpose, a simple magnetic stirrer or an electrically operated agitator may be used. Mixing can also be effected using a liquid-liquid homogenizer or emulsifier. Depending on the viscosity of the resultant mixture, the time required to achieve a homogenous mass may range from 15 minutes to 1 hour. For those nutrients which are sensitive to atmospheric oxidation, the mixing step can be performed under an inert atmosphere or in the presence of an antioxidant stabilizer.

For those nutrients wherein a faster dissolution is required in the gastrointestinal tract, one can optionally incorporate a food grade surfactant to enhance the solubility of the lipophilic nutrient and its bioavailability.

The homogenous mass so obtained is then subjected to a step of heating under a reduced pressure. Since vast majority of the lipophilic nutrients are sensitive to the heat, light and oxygen, it may be necessary to carry out such heating at low temperatures, preferably not exceeding 45 deg C. It would also be preferable to heat under an inert atmosphere using inert gases such as nitrogen or argon. The process of heating is continued until the resultant dispersion has less than 25 parts per million of the solvent.

After ensuring the reduction of solvent residues at less than 25 parts per million, the resultant molecular dispersion is allowed to cool to ambient temperature and then it is passed through 100 mesh sieve to remove any agglomerate or lumps The homogenous mass is then filled in to appropriate containers.

### BRIEF DESCRIPTION OF THE FIGURES

FIG. 1 shows a graph of the effects of treatment on fasting plasma glucose in STZ-induced diabetic rats.

FIG. 2 shows a graph on the delay of diabetic cataract in rats by treatment.

FIG. 3 shows the SDS-PAGE pattern of soluble fraction of lens proteins.

FIG. 4 shows a graph on size exclusion chromatography of lens protein soluble fraction.

FIG. 5 shows a graph on spectrofluoremetric measurement of lens sorbitol.

FIG. 6 shows a graph on HPLC measurement of plasma lutein levels.

FIG. 7 shows representative wave forms of oscillatory potentials (OPs) from different groups and total amplitudes.

FIG. 8 shows representative histology of retina.

FIG. 9 shows expression of Rhodopsin by real-time PCR (A) and immunohistochemistry (B).

FIG. 10: Expression of NGF by real-time PCR (A) and immunohistochemistry (B).

FIG. 11 shows expression of VEGF by immunoblotting.

FIG. 12 shows expression of PDGF by immunohistochemistry.

FIG. 13 shows serum lutein levels measured by RP-HPLC.

FIG. 14 shows representative wave forms of OPs from individual animals of different groups and total amplitudes.

FIG. 15 shows representative histology of retina.

FIG. 16 shows expression of rhodopsin by real-time PCR (A) and immunohistochemistry (B).

FIG. 17 shows expression of NGF by real-time PCR (A) and Immunohistochemistry (B).

FIG. 18 shows expression of VEGF by immunoblotting.

FIG. 19 shows expression of PDGF by immune histochemistochemistry

### DETAILED DESCRIPTION

Diabetes mellitus can cause a variety of eye problems, the most common being diabetic retinopathy (DR) and diabetic cataract which are the most common causes of blindness. Antioxidant compounds are considered to have high antioxidant potential in the prevention of many human ailments such as age related macular degeneration, cataract, diabetic eye complications and various other diseases.

Lutein is a naturally occurring antioxidant found in green leafy vegetables like spinach. Lutein is also found in eye mainly present in macula lutea. It is well known that lutein is a carotenoid and powerful antioxidant. It has been used to treat cataracts and macular degeneration which is an age related degenerative disorder. Lutein has also shown protective antioxidant activity in human HepG2 cell lines.

Zeaxanthin is one of the most common carotenoid alcohols found in nature. Lutein and zeaxanthin have identical chemical formulas and are isomers, but they are not stereoisomers. The only difference between them is in the location of the double bond in one of the end rings. This difference gives lutein three chiral centers whereas zeaxanthin has two. Because of symmetry, the (3R,3′S) and (3S,3′R) stereoisomers of zeaxanthin are identical. Therefore, zeaxanthin has only three stereoisomeric forms. The (3R,3′S) stereoisomer is called meso-zeaxanthin.

The conjugated double bonds of lutein and zeaxanthin contribute to the distinctive colors of each pigment, and also influence the ability of these to quench singlet oxygen. Due to the extra conjugated double bond, zeaxanthin is believed to be a stronger anti-oxidant compared to lutein. It has been demonstrated that the complex of lutein and zeaxanthin isomers act as a better antioxidant than the free xanthophylls, facilitating improved protection from oxidative damages.

Curcumin, a yellow pigment from Curcuma longa, is a major component of turmeric and is commonly used as a spice and food-coloring agent. It is also used as a cosmetic and in some medical preparations. The desirable preventive or putative therapeutic properties of curcumin have also been considered to be associated with its antioxidant and anti-inflammatory properties. Curcumin is thought to play a vital role against a variety of chronic pathological complications such as cancer, atherosclerosis, and neurodegenerative diseases.

The lipophilic nutrients are poorly absorbed if administered either as oil suspensions or as beadlets, which are the currently used forms. The main reason for poor absorption is their poor solubility in water. Due to their insolubility their bioavailability is very poor. Dispersion of nutritional products at molecular level provides higher efficiency for micellization of nutrients in water and thereby increases the bioavailability.

Compositions herein of lipophilic nutrients contain at least 80% by weight of total xanthophylls, out of which the trans-lutein content is 80-95% w/w; (R,R)-zeaxanthin is 14-20% w/w; (R,S)-zeaxanthin is 0.01-1% w/w, or trans-lutein content is 80-95% w/w; (R,R)-zeaxanthin is 14-20% w/w, and traces of other carotenoids derived from the plant extracts/oleoresin containing xanthophylls/xanthophylls esters, or curcumin which contains 5-95% of curcuminoids in highly water soluble form, and have enhanced bioavailability in delaying the development and maturation of eye related complications of diabetes.

Compositions herein comprise lipophilic nutrients; stabilizer; water soluble hydrophilic carrier; and optionally a surfactant.

Compositions herein contain at least 80% by weight of total xanthophylls, out of which the trans-lutein content is 80-95% w/w; (R,R)-zeaxanthin is 14-20% w/w; (R,S)-zeaxanthin is 0.01-1% w/w, or trans-lutein content is 80-95% w/w; (R,R)-zeaxanthin is 14-20% w/w, and traces of other carotenoids derived from the plant extracts/oleoresin containing xanthophylls/xanthophylls esters, or curcumin which contains 5-95% of curcuminoids.

The stabilizer used is selected from ascorbic acid, butylated hydroxyanisole (BHA), butylated hydroxytoluene (BHT), ascorbyl palmitate, rosemary extract, mixed natural tocopherols, alpha tocopheryl acetate, sodium ascorbate, castor oil derivatives, sodium lauryl sulfate and mixtures thereof.

The carrier used is selected from polyethylene glycol 200, polyethylene glycol 400, ethylene glycol, propylene glycol, glycerol, sorbitol, glucose syrup, corn steep liquor, mannitol, polyethylene glycol 6000, polyethylene glycol 10000, Polyethylene glycol 20000, polyvinyl pyrrolidone, hydroxyl propyl methyl cellulose, sucrose, glucose, sodium chloride, hydroxyl propyl cellulose, polyvinyl alcohol, soluble starch, hydrolyzed starch, and mixtures thereof.

The surfactant is selected from polysorbate 20, polysorbate 60, polysorbate 80, lecithin, sucrose fatty acid esters, glyceryl fatty acid esters, sodium lauryl sulfate, and mixtures thereof.

Studies with rats were carried out to test the activity of lipophilic nutrients in diabetic eye complications with four samples viz water soluble compositions of trans-lutein and zeaxanthin isomers (sold under the brand name UltraSol Lutemax2020™); concentrates containing trans-lutein and zeaxanthin isomers (sold under the brand name Lutemax2020 ; and water soluble compositions containing curcumin (sold under the brand name UltraSol CurcuWin™) and curcumin powder.

In some embodiments, the compositions herein include xanthophyll compositions containing macular pigments of trans-lutein and zeaxanthin isomers, including (R,R)-zeaxanthin and (R,S)-zeaxanthin derived from the plant extract/oleoresin containing xanthophylls/xanthophylls esters which is safe for human consumption and useful for nutrition and health care.

In some embodiments, a xanthophyll composition contains at least 80% by weight of total xanthophylls of which the trans-lutein content is at least 80% by weight and the remaining being zeaxanthin isomers, including (R,R)-zeaxanthin and (R,S)-zeaxanthin derived from the plant extract/oleoresin containing xanthophylls/xanthophylls esters which is safe for human consumption and useful for nutrition and health care.

In some embodiments, a xanthophyll composition contains at least 85% by weight of total xanthophylls, out of which the trans-lutein content is at least 85% by weight and the remaining being zeaxanthin isomers, including (R,R)-zeaxanthin and (R,S)-zeaxanthin derived from the plant extract/oleoresin containing xanthophylls/xanthophylls esters which is safe for human consumption and useful for nutrition and health care.

In some embodiments, a xanthophyll composition contains at least 85% by weight of total xanthophylls, out of which at least 80% by weight being trans-lutein, at least 6% by weight being (R,R)-zeaxanthin and at least 6% by weight being (R,S)-zeaxanthin derived from the plant extract/oleoresin containing xanthophylls/xanthophylls esters which is safe for human consumption and useful for nutrition and health care.

In some embodiments, a xanthophyll composition contains at least 85% by weight trans-lutein and at least 4% by weight (R,R)-zeaxanthin and at least 5% by weight (R,S)-zeaxanthin derived from the plant extract/oleoresin containing xanthophylls/xanthophylls esters which is safe for human consumption and useful for nutrition and health care.

In some embodiments, a xanthophyll composition contains at least 85% by weight of total xanthophylls, out of which at least 80% by weight being trans-lutein, the remaining 15% by weight being zeaxanthin isomers, including (R,R)-zeaxanthin and (R,S)-zeaxanthin derived from the plant extract/oleoresin containing xanthophylls/xanthophylls esters which is safe for human consumption and useful for nutrition and health care.

In some embodiments, a process for the preparation of the xanthophyll composition containing macular pigments consisting of trans-lutein, zeaxanthin isomers, namely (R,R)-zeaxanthin and (R,S)-zeaxanthin derived from the plant extract/oleoresin containing xanthophylls/xanthophylls esters which is safe for human consumption and useful for nutrition and health care, in which:

a) the saponification step to convert xanthophyll esters present in plant extract/oleoresin into the de-esterified form can be combined with limited isomerization of lutein to produce xanthophyll composition containing higher amount of trans-lutein, the remaining being zeaxanthin isomers, including (R,R)-zeaxanthin and (R,S)-zeaxanthin and traces of other carotenoids derived from the plant extract/oleoresin containing xanthophyll/xanthophylls esters which is safe for human consumption and useful for nutrition and health care;

b) in the saponification step, potassium hydroxide or sodium hydroxide can be dissolved in 1-propanol without the addition of water;

c) the temperature of the saponification/isomerization can be between 70 to 100 Deg C. preferably around at 95 degree and the period of saponification can be 1-2 hr.; and

d) the ethyl acetate employed in the process can be recovered and used if required, thereby making the process economical.

In some embodiments, a xanthophyll composition contains macular pigments of trans-lutein, zeaxanthin isomers, including (R,R)-zeaxanthin and (R,S)-zeaxanthin, derived from the plant extract/oleoresin containing xanthophylls/xanthophylls esters which is safe for human consumption and useful for nutrition and health care which comprises of at least 80% by weight is total xanthophylls, of which the ratio of trans- lutein and zeaxanthin isomers being in the range of 4:1 to 6:1 and the ratio of the isomers of zeaxanthin being in the range of 80:20 to 20:80. In some embodiments, the ratio of trans-lutein and zeaxanthin isomers is at a ratio of about 5:1.

In some embodiments, a xanthophyll composition contains at least 85% by weight of total xanthophylls, of which the trans-lutein content is at least 85% and the ratio of trans-lutein and zeaxanthin isomers being in the range of 4:1 to 6:1 and the ratio of the isomers of zeaxanthin being in the range of 80 to 20: 20 to 80

In some embodiments, a process for the preparation of a xanthophyll composition containing macular pigments of trans-lutein and zeaxanthin isomers, including (R, R)-zeaxanthin and (R,S)-zeaxanthin derived from the plant extract/oleoresin containing xanthophylls/xanthophylls esters which is safe for human consumption and useful for nutrition and health care, which comprises:

(a) saponifying and partially isomerising simultaneously the xanthophyll esters present in the plant extract/oleoresin containing xanthophyll esters by admixing the extract/oleoresin with alkaline solution of 1-propanol, the ratio of alkali and 1-propanol being in the range of 1:0.5 to 1:1 by weight/volume, heating the resultant mass at a temperature in the range 70-100 degree C., preferably 95 Deg C. for a period in the range of 1 to 5 hrs to obtain a saponified/isomerised crude concentrate;

(b) admixing the resultant saponified/isomerised crude concentrate obtained in step (a) with water, the ratio of the concentrate and water used being in the range from 1:2 to 1:3 volume/volume, to form a diluted oily mixture;

(c) extracting the diluted oily mixture obtained in step (b) with ethyl acetate, the ratio of diluted oily mixture and ethyl acetate used being in the range of 1:1.5 to 1:2 volume/volume to get an extract containing the xanthophyll composition;

(d) evaporating the composition obtained in step (c) to remove ethyl acetate;

(e) purifying the composition resulting from step (d) by washing first with non polar and later with polar solvents and filtering;

(f) drying the resulting composition under vacuum at a temperature in the range of 40 to 45 Deg C for a period ranging from 48-72 hours;

(g) if desired recovering the ethyl acetate used in step (c) by conventional methods and if required reused; and

(h) if desired storing the resulting composition in an inert atmosphere at −20 Deg.

By adjusting the temperature, period and the amount of alkali in the step (a) the ratios in steps (b) and (c), the desired composition can be obtained

It is to be noted that leafy and green vegetables, corn, fruits and/or marigolds may be used as the source for the xanthophylls oleoresin. But considering that lutein is present along with zeaxanthin in free form associated with large amounts of chlorophyll and other undesirable carotenoids in most of the fruits, though according to the present invention the use of leafy and green vegetables corn, fruits is possible and considering the low concentration of lutein and zeaxanthin in the above materials and further the elaborate steps of purification which is required being not economical, marigold is the preferred choice as the starting material for the preparation of the composition of the present invention

Specifically, commercially available food grade marigold oleoresin produced by hexane extraction can be used as starting material (Kumar et. al Process for the Preparation of Xanthophylls Crystals, U.S. Pat. No. 6,743,953, 2004; Kumar U.S. Pat. No. 6,737,535, 2004) for the preparation of the xanthophyll composition comprising of trans-lutein, and zeaxanthin isomers.

Marigold flower (Tagetes erecta) is considered to be the best possible commercial source for trans-lutein as it contains lutein mono- and diesters as the major carotenoid constituents. The alkali used in step (a) may be selected from sodium hydroxide or potassium hydroxide.

The non-polar solvent used in step (d) may be a hydrocarbon solvent which may be selected from pentane, hexane and heptane, and the like, preferably hexane. The polar solvent used in step (e) may be selected from a lower aliphatic alcohol.

The inert atmosphere used for storing the resulting composition may be maintained inert gas like nitrogen.

In some embodiments, the extract containing xanthophyll ester is mixed with 1-propanol in which alkali is already dissolved. The ratio of alkali to 1-propanol and the plant extract is 0.5-1:0.5-1.0 and 1.0 respectively. The mixture is heated to a temperature of 90 degree C. and maintained for 1-5 hours, under agitation. The total xanthophylls in the reaction mixture are determined by Spectrophotometric analysis (AOAC-16th Edition Method 970.64) while the HPLC analysis of the same provides the percentage of trans- lutein and zeaxanthin. (Hadden et al., J. Agric. Food. Chem, 47, 4189-494, 1999).

The saponification of the extract/oleoresin results in the liberation of xanthophylls in free form along with alkali salts of fatty acids. The isomerization reaction converts part of the lutein from marigold into (R, S)-zeaxanthin. The isomerization of lutein to zeaxanthin isomers can be varied by changing process parameters such as alkali:solvent ratio, temperature and duration. The composition of the xanthophylls in the reaction mixture is analyzed by extracting into hexane:acetone:ethanol:toluene (10:7:6:7 v/v) followed by addition of hexane and 10% sodium sulphate solution and analyzing the upper layer by HPLC.

After obtaining the desired degree of isomerization and the xanthophylls composition with trans-lutein content typically around 85%, the reaction mixture is diluted with water and stirred well at room temperature to obtain a yellow oily layer containing xanthophylls in free form associated with fatty acid, soaps and impurities.

After transferring this oily layer into a separatory funnel, ethyl acetate is added and the xanthophylls extracted. The ethyl acetate layer is washed twice with an equal volume of de-ionized water. Thus, the fatty acids and soapy materials are removed into water which is then discarded. The ethyl acetate extract is concentrated by distilling off the solvent under reduced pressure to recover ethyl acetate and the crude xanthophyll concentrate.

The xanthophyll concentrate composition is subjected to purification by agitating with hexane at room temperature for one hour, followed by filtration. The xanthophyll mass is further washed with ethanol and the resulting orange crystals is dried under vacuum at ambient temperature for 72 hours.

In some embodiments, the composition is a water-soluble composition having enhanced bioavailability comprises a synergistic combination of curcumin, at least an antioxidant, a hydrophilic carrier and a fat.

In some embodiments, a process for the preparation of the curcumin composition which comprises the steps of dissolving curcumin, at least one antioxidant, a hydrophilic carrier and a fat in a solvent to form a homogenous mass; warming the resultant mass at a temperature ranging from 25° C. to 60° C. for a period of 4 to 8 hours to obtain a dry wet mass; removing the solvent by evaporation to form dry mass and pulverizing the dry mass to form a fine powder.

In some embodiments, a water-soluble composition has enhanced bioavailability containing curcumin which is available in an orally administrable form.

In some embodiments, a water-soluble composition has enhanced bioavailability containing curcumin, which is safer for human consumption without any significant side effects.

In some embodiments, a process is described for the preparation of a water-soluble composition containing curcumin having enhanced bioavailability,

In some embodiments, a water-soluble composition has enhanced bioavailability which comprises a synergistic combination of curcumin, at least an antioxidant, a hydrophilic carrier and a fat.

In some embodiments, a process for the preparation of a novel water-soluble composition having enhanced bioavailability comprises:

(i) dissolving curcumin, at least one antioxidant, a hydrophilic carrier and a fat in a solvent to form a homogenous mass;

(ii) warming the resultant mass at a temperature ranging from 25° C. to 60° C. for a period of 4 to 8 hours to obtain a dry wet mass;

(iii) removing the solvent by evaporation to form dry mass and

(iv) pulverizing the dry mass to form a fine powder.

Curcumin used in the step (i) can be commercially available one with an assay ranging between 85-96%. It can also be an extract of turmeric rich in curcumin. The amount of curcumin added may be sufficient to produce a water soluble curcumin with an assay of 1-55% curcumin.

The antioxidants used in step (i) can be selected from natural tocopherols, ascorbyl palmitate, rosemary extract, epigallocatechin gallate, catechins, ascorbic acid and mixture thereof. The amount of antioxidant used may range between 1-10%.

The hydrophilic carrier used in the step (i) can be selected from soluble starch, hydroxy propyl methyl cellulose, sodium carboxy methyl cellulose, polyvinyl pyrrolidone, polyethylene glycols 200-20000, glycerol, sorbitol, mannitol, glucose, sugar and mixture thereof. The quantity of hydrophilic carrier added may range between 10-90%.

The fat used in the step (i) may be selected from milk fat, medium chain triglycerides, long chain triglycerides, hydrogenated vegetable oils, and mixtures thereof. The quantity of fat used may range from 1-25%.

The solvent used for dissolving in the step (i) may be selected from isopropyl alcohol, acetone, methanol, alcohol, and mixtures thereof. The temperature maintained for obtaining an homogenous mass may range from ambient to 70 deg C.; preferably 25° C. to 60° C.

The removal of solvent in step (ii) can be performed in vacuum distillation or evaporation technique, or by spray drying technique. The resultant dry mass is pulverized by using mortar and pestle, mixer-grinder, multi-mill, ball mill, jet mill and the like.

The beneficial effects of curcumin have been well known. However, there are many problems associated with the bioavailability of curcumin when delivered in the oral form. Major portion of ingested curcumin is excreted through the feces unmetabolized and the small portion that gets absorbs is converted into other metabolites and excreted. Curcumin does not easily penetrate the gastrointestinal tract and is subject to liver and other intestinal enzymes. Owing to these enzymes, the curcumin within the body is rapidly metabolised thus reducing its bioavailability in the body. The small amount of curcumin that enters the bloodstream is rapidly metabolized by the liver and kidney. Therefore, although curcumin is highly lipophilic (and so easily crosses the blood brain harrier), only very small amounts of orally administered curcumin are registered in the serum and in the brain tissue.

Cytochrome P450 is a phase I metabolizing isoenzyme which is required for metabolizing toxic chemicals such as heterocyclic amines to induce DNA adduct formation leading to carcinogenesis. Curcumin when ingested in the body enters the gastrointestinal tract and is found to inhibit Cytochrome P450. As mentioned hereinabove, there have been studies carried out to increase the bioavailability of curcumin when used along with piperine. Piperine is a bioenhancer which inhibits Cytochrome P450 and thereby prevents metabolism of curcumin in the body. Compositions herein are seen to enhance the bioavailability without the presence of any additional bioenhancer,

The water soluble compositions of curcumin comprises of an antioxidant, a hydrophilic carrier and a fat. The antioxidant along with curcumin inhibits the Cytochrome P450. On the other hand, the presence of fat coating on the composition prevents the composition from attack by liver microsomal or other intestinal enzymes as these enzymes attack only aqueous compounds. Thus, the antioxidant and the fat play a vital role in enhancing the bioavailability of curcumin.

Earlier studies demonstrated that Lutemax2020® delayed diabetic cataract in rats at 1% in the diet but not at (0.1%). Further Lutemax2020® (1%) only delayed but did not completely prevent diabetic cataract and hence the water soluble composition of trans-lutein and zeaxanthin isomers (UltraSol Lutemax2020™) was used to test further the effects of the compositions in the prevention/treatment of diabetic eye complications, such as diabetic cataract and diabetic retinopathy.

The following examples are given by the way of illustration of the present innovations and therefore should not be construed to limit their scope.

To determine the effect of water soluble composition containing trans-lutein and zeaxanthin isomers and water soluble composition containing curcumin in comparison to regular compositions containing trans-lutein and zeaxanthin isomers and regular compositions containing curcumin in preventing or delaying diabetic cataract and diabetic retinopathy the rats were induced diabetes by using streptozotocin (STZ).

### Example 1

### Effect of UltraSol Lutemax2020™ and UltraSol CurcuWin™ in Diabetic Cataract

Experimental Design

Male Wistar strain (WNIN) rats (2 months old; Average BW of 213±14 g) obtained from the National Center for Laboratory Animal Sciences, National Institute of Nutrition, Hyderabad, India (NCLAS, NIN). Animals were maintained at NCLAS, NIN and kept for acclimatization in an experimental room for two weeks. Diabetes was induced in overnight fasted animals by a single intraperitoneal injection of STZ (30 mg/kg) in 0.1 M citrate buffer, pH 4.5. Another set of rats, which received only a vehicle, served as the control (Group I; n=12). Fasting blood glucose levels were measured 72 h after STZ injection. Animals having blood glucose levels >150 mg/dL were considered diabetic and those were only divided into five groups (Group II-VI). A group of control rats (n=6) were fed with 0.01% soluble curcumin (Group VII) and soluble 0.5% lutein alone (Group VIII).

All the animals were housed in individual cages maintained on their respective diets for 12 weeks and drinking water was provided ad libitum throughout the study period.

TABLE 1
Experimental groups and diets

| | | Number | |
|---|---|---|---|
| | Group | of | Diet |
| I | Control | 12 | American Institute of Nutrition (AIN) 93 |
| II | Diabetic | 14 | AIN 93 |
| III | Diabetic + | 12 | AIN 93 with soluble curcumin (SC) 0.01% |
| | SC | | |
| IV | Diabetic + | 12 | AIN 93 with regular curcumin (RC) 0.01% |
| | RC | | |
| V | Diabetic + | 12 | AIN 93 with soluble lutein (SL) 0.5% |
| | SL | | |
| VI | Diabetic + | 12 | AIN 93 with regular lutein(RL) 0.5% |
| | RL | | |
| VII | Control + | 6 | AIN 93 with soluble curcumin (SC) 0.01% |
| | SC | | |
| VIII | Control + | 6 | AIN 93 with soluble lutein (SL) 0.5% |
| | SL | | |

Animal Care: Institutional and national guidelines for the care and use of animals were followed and all experimental procedures involving animals were approved by the IAEC (institutional animal ethical committee) of National Institute of Nutrition.

Animals were housed in individual cages in a temperature (22° C.) and humidity controlled room with a 12-h light/dark cycle. All the animals had free access to water.

Food intake (daily) and body weights (weekly) were monitored.

Slit lamp examination and Cataract grading: Eyes were examined every week using a slit lamp bio microscope on dilated pupils. Initiation and progression of lens opacity was graded into five categories (0-4).

Mortality: During the course of the study, 3 animals in Group II and 2 animals each from Groups III-VI have died to hyperglycemia as expected.

Blood/Lens collection and processing: Blood was drawn once in every week from retro orbital plexus for glucose and insulin estimation. At the end of 12 weeks, animals were sacrificed by CO2 asphyxiation and lenses were dissected by posterior approach and stored at −70° C. until further analysis. A 10% homogenate was prepared from 3-5 pooled lenses in 50 mM phosphate buffer, pH 7.4. All the biochemical parameters were analyzed in the soluble fraction of the lens homogenate (15,000x g at 4° C.) except for lens malondialdehyde (MDA), which was determined in the total homogenate.

Biochemical estimations: Lens MDA, as thiobarbituric acid reacting substances (TBARS) protein carbonyl content were determined according to the methods for estimating the protein carbonyl content described by Suryanarayana P et al. in the research paper “Curcumin and turmeric delay streptozotocin-induced diabetic cataract in rats,” Invest Ophthalmol Vis Sci 2005; 46(6):2092-9. Total, soluble and insoluble protein was assayed by Lowry method using bovine serum albumin (BSA) as standard.

Plasma lutein levels: Plasma lutein levels were measured by HPLC using 4.6×150 mm, 5 μm, spherisorb waters C18 column connected to Dionex UltiMate 3000 Rapid Separation Liquid Chromatography (RSLC). The column was equilibrated with mobile phase, isocratic solvent mixture of acetonitrile: dichloroethane: methanol in a ratio of 70:20:10 (v/v) at flow rate of 0.5 ml/min at 25° C. 2 μl of plasma samples (extracted with hexane) were loaded onto the column and lutein was detected at 300-600 nm.

Sodium dodecyl sulfate polyacrylamide gel electrophoresis (SDS-PAGE) and size exclusion chromatography of lens proteins: Subunit profile and cross-linking of soluble proteins were analyzed on 10% polyacrylamide in the presence of sodium dodecyl sulfate (SDS) under reducing conditions. Crystalline distribution in the soluble protein fraction was performed by size exclusion chromatography on a 600×7.5 mm TSK-G4000 SW column (TOSOH Co., Japan) using a HPLC system. The column was equilibrated with 0.1 M sodium phosphate buffer pH 6.7 containing 0.1 M sodium chloride at a flow rate of 1 ml/min.

Statistical analysis: One-way analysis of variance (ANOVA) was used for testing statistical significance between groups of data and individual pair difference was tested by means of Duncan's multiple-range test. Heterogeneity of variance was tested by the nonparametric Mann Whitney test where p<0.05 was considered as significant.

Results

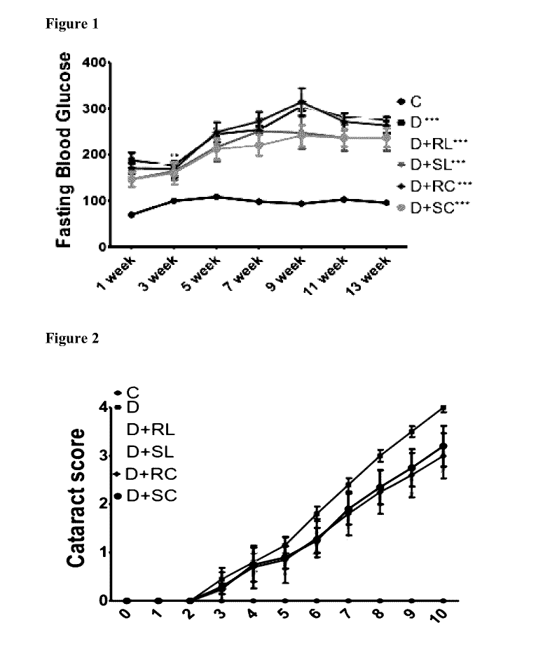

Fasting blood glucose: FIG. 1 summarizes the results of fasting plasma glucose in different groups of animals throughout the treatment period. The plasma glucose concentrations of the diabetic control rats were significantly higher than those of the non-diabetic control rats throughout the experiment. Though there was lower mean fasting plasma glucose levels observed in groups treated with SC and SL, but no significant effect of treatment on plasma glucose in diabetic rats was observed.

FIG. 1: Effects of treatment on fasting plasma glucose in STZ-induced diabetic rats. (FIG. 1 is shown in the drawings accompanied with the specification). The data is expressed as mean±standard error of measurement (SEM). Control (Non-diabetic control); D (Diabetic control); D+RL (diabetic+regular lutein); D+SL (Diabetic+soluble lutein); D+RC (Diabetic+regular curcumin); D+SC (Diabetic+soluble curcumin) ***=p<0.001.

Cataract development and progression: Onset and progression of cataract is monitored by slit lamp biomicroscope examination as described below: Eyes were examined every week using a slit lamp biomicroscope (Kowa SL15, Portable, Japan) on dilated pupils.

Initiation and progression of lenticular opacity was graded into five categories as follows: “clear”, clear lenses and no vacuoles present; “stage 1”, vacuoles cover approximately one half of the surface of the anterior pole, forming a subcapsular cataract; “stage 2”, some vacuoles have disappeared and the cortex exhibits a hazy opacity; “stage 3”, a hazy cortex remained and dense nuclear opacity is present; and “stage 4”, a mature cataract is observed as a dense opacity in both cortex and nucleus (FIG. 2).

FIG. 2: Delay of diabetic cataract in rats by treatment. (FIG. 2 is shown in the drawings accompanied with the specification). The data is expressed as mean±SEM. Control (Non-diabetic control); D (Diabetic control); D+RL (diabetic+regular lutein); D+SL (Diabetic+soluble lutein); D+RC (Diabetic+regular curcumin); D+SC (Diabetic+soluble curcumin)

The onset of cataract due to hyperglycemia was observed in diabetic animals after three weeks of STZ, injection. The average incidence of cataract was calculated and presented in FIG. 2. Though there was no delay in the onset there is a clear delay in the progression and maturation of cataract in all the treatment groups when compared to Group-D. Group-D animals showed lens opacification (stage-IV) by the end of the 10^{th} week, while the treatment groups showed around stage-2.5 to 3. The data clearly indicates there is a significant delay in the progression and maturation of cataract intervention groups from the sixth week onwards, when compared to group-D. At the end of ten weeks, the severity of cataracts was significantly lower in groups D+RL (stage 3.1), D+SL (stage 2.7), D+RC (stage 3.0) and D+SC (stage 3.2) than in Group-D (Stage 4), indicating that intervention with any agent delayed the maturation of diabetic cataract due to slow progression, Further SL seems to be more effective than RL, but SC did not show superiority in efficacy over RC in progression of cataract. All the lenses in Group-C during the entire experimental period appeared to be normal, clear and free of opacities.

Lens Biochemical Analysis:

Individual lenses were weighed and pooled into 4 lenses for a pool, and such 4-5 pools were formed per group. A 10% homogenate was prepared in 50 mM sodium phosphate buffer pH 7.4, with tissue homogenizer at intermittent time gaps to avoid excess heat generation. Separate aliquots of total homogenate (TH) were made 250 μl for TBARS assay, 150 μl for sorbitol estimation, and 20 μl for protein estimation. Remaining homogenate was centrifuged at 10,000 RPM for 30 min at 4° C. Supernatant was separated into labelled vials, as total soluble protein (TSP).

Determining soluble percentage of protein in lens homogenate: Protein estimation was done in lens homogenate and soluble fraction by Lowry's method. Amount of protein present per gram weight of lens was calculated. Percentage of soluble protein was calculated by multiplying the fraction of soluble protein by 100.

We analyzed the total and soluble protein content in the lenses of all the experimental groups. There was a significant decrease in both total and soluble protein in Group-D compared with the control group. This could be due to a partial leakage of proteins into the aqueous humor, or aggregation of proteins and insolubilization. Among the treatment groups, SL and RC significantly prevented loss of soluble protein compared to group-D, whereas, SL alone had shown significant difference against group D in percentage of soluble protein. SC and RL had shown partial beneficial effect in preventing insolubilization of lens proteins but were relatively less significant statistically.

TABLE 2
Protein content in total and soluble fraction of lens homogenate.
The data is expressed as mean ± SEM.

| | Total protein | Soluble protein | Percentage |
|---|---|---|---|
| Group | (mg/gm lens) | (mg/gm lens) | soluble protein |
| Control (C) | 516.54 ± 9.3 | 388.77 ± 8.2 | 75.19 ± 2.2 |
| D | 281.34 ± 31.0*** | 128.87 ± 11.0*** | 46.87 ± 2.5*** |
| D + RL | 311.79 ± 7.8 | 156.55 ± 4.7 | 50.35 ± 1.9 |
| D + SL | 376.53 ± 20.9 | 218.78 ± 14.3 ^{##} | 58.04 ± 1.8 ^{#} |
| D + RC | 368.50 ± 19.2 | 202.63 ± 21.6 ^{#} | 54.43 ± 3.8 |
| D ± SC | 351.83 ± 41.5 | 178.99 ± 25.9 | 50.16 ± 1.3 |

n = 6;
Control (Non-diabetic control);
D (Diabetic control);
D + RL (diabetic + regular lutein);
D + SL (Diabetic + soluble lutein);
D + RC (Diabetic + regular curcumin);
D + SC (Diabetic + soluble curcumin);
***= p < 0.001,
** = P < 0.01 and
*= P < 0.05 Vs C;
^{##} = P < 0.01 and
^{#} = P < 0.05 Vs D

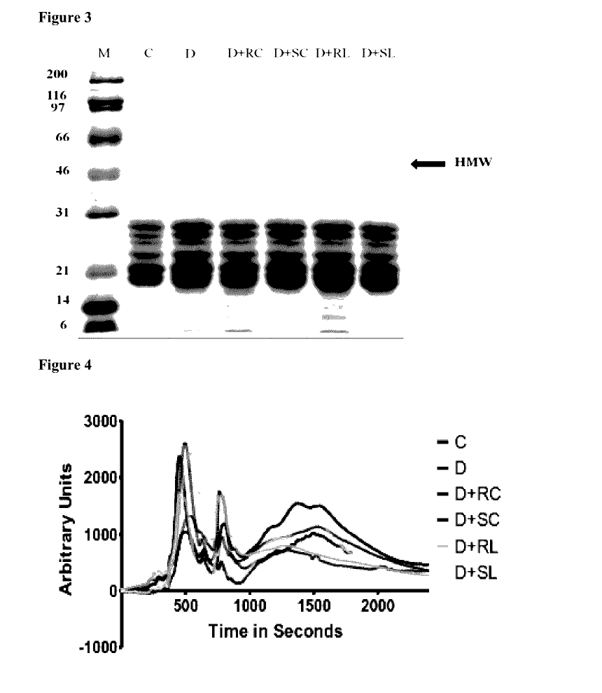

SDS-PAGE protein profiling: Differences in protein distribution pattern were observed by running the lens protein samples on 12% polyacrylamide gel. 30 μg of protein was loaded per well along with molecular weight marker for SDS-PAGE (Broad range SDS-PAGE marker, BioRad). The SDS-electrophoretic pattern of the soluble protein fraction showed a band corresponding to aggregated proteins at ˜50 kDa in group-D in relation to the group-C and with reduced band intensity in treatment groups RL, SL, RC and SC. FIG. 3 represents the SDS-PAGE pattern of soluble fraction of lens proteins, as shown in the drawings accompanied with the specification.

Size Exclusion High Performance Liquid Chromatography:

Size exclusion chromatography on TSK-3000 HPLC column resulted in resolution of crystalline lens proteins. HPLC profile demonstrated the reduced peak area in low molecular weight region, and increased peak area in the high molecular weight protein region in Group-D (red line) TSP, as compared to group-C (black line). This suggests there was a phenomenon of protein aggregation in diabetic conditions. Intervention with all except RL, i.e. SL, RC and SC normalized the profile of TSP. Except RL, all other compositions SL, RC and SC intervened with TSP and normalized the profile, while RL did not show any effect on TSP levels and they remained abnormal like diabetic condition.

FIG. 4 represents the size exclusion chromatography of lens protein soluble fraction, as shown in the drawings accompanied with the specification.

SORBITOL LEVELS: We assessed accumulation of sorbitol in the lens of all experimental animals and the data were presented in the FIG. 8. Group-D showed significantly elevated levels of sorbitol (5.877±0.27) when compared with group-C (0.301±0.04 u moles/gm lens of sorbitol). Among the intervention groups, except SC, remaining treatments did not lower sorbitol accumulation compared to group-D. Group-SC showed significantly lower sorbitol levels when compared to group-D and significantly higher sorbitol levels when compared with group-C. This might be attributed to the additional pharmacological action of curcumin as an aldose reductase inhibitor.

FIG. 5: Spectrofluoremetric measurement of lens sorbitol (FIG. 5 is shown in the drawings accompanied with the specification). The data is expressed as mean±SEM. n=6; Control (Non-diabetic control); D (Diabetic control); D+RL (diabetic+regular lutein); D+SL (Diabetic+soluble lutein); D+RC (Diabetic+regular curcumin); D+SC (Diabetic+soluble curcumin); *=P<0.05 Vs C; #=P<0.05 Vs D.

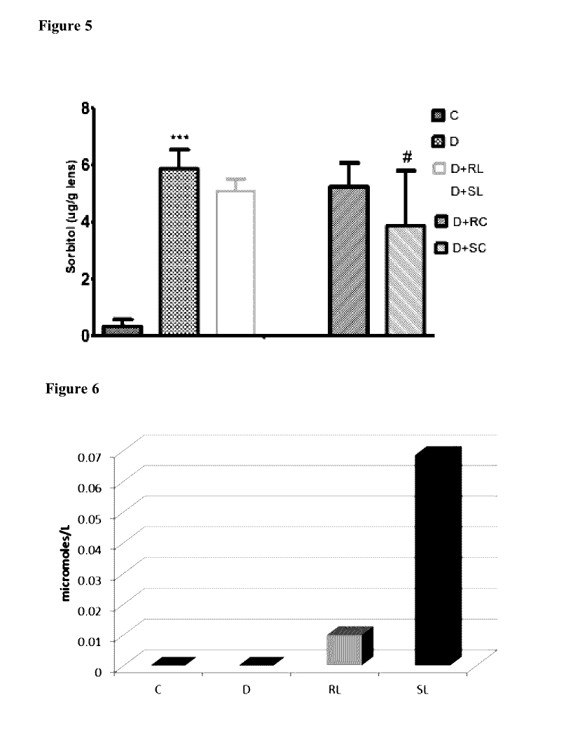

Plasma lutein levels: Plasma lutein levels were measured by HPLC. Since rodent chow diet does not contain carotenoids, lutein was not detected in control and diabetic rats. However, lutein could be detected in lutein supplemented groups. Feeding of diabetic rats with regular lutein results in 0.01 micromoles/L of plasma lutein (FIG. 6). Feeding of soluble lutein led to seven fold increase in plasma lutein 0.07 micromoles (FIG. 6), suggesting that soluble lutein increases bioavailability of lutein significantly and may be the reason for improved beneficial effects with soluble lutein compared to regular lutein.

FIG. 6: HPLC measurement of plasma lutein levels (FIG. 6 is shown in the drawings accompanied with the specification). The data was expressed as mean±SEM. n=6; Control (Non-diabetic control); D (Diabetic control); D+RL (diabetic+regular lutein); D+SL (Diabetic+soluble lutein).

Conclusion

Supplementation of curcumin rescued photoreceptor degeneration in transgenic rats with P23H rhodopsin mutation. Feeding of dietary antioxidant curcumin was effective in delaying streptozotocin (STZ)-induced diabetic cataract in rats mainly through its antioxidant property. In addition, curcumin inhibited diabetes-induced expression of vascular endothelial growth factor (VEGF) in rat retina and lens aldose reductase (AR).

Soluble lutein is more effective in delaying diabetic cataract compared to regular lutein at dose of 0.5% in the diet which is reflected in molecular analysis related to cataract genesis. Increased bioavailability of soluble lutein might explain the observed biological effects of soluble lutein compared to regular lutein. The efficacy of soluble curcumin was almost comparable with regular curcumin.

### Example 2

### Effect of UltraSol Lutemax2020™ in Diabetic Retinopathy by Nutrigenomics Approach

The effect of soluble lutein (UltraSol Lutemax2020™) was investigated in diabetic rats with respect to its beneficial effect on retina by a nutrigenomics approach and the effect was compared with regular lutein)(Lutemax2020®.

Methodology

Animal model: The streptozotocin (STZ) rat model of diabetes has been one of the most commonly used models of human disease with respect to diabetes. It is known to mimic many of the acute and some of the chronic complications observed in human diabetes. This model has the advantage of being highly reproducible and the timelines for various complications to develop are well recognized and reproducible. Given the established similarities of some of the structural, functional and biochemical abnormalities of human disease, it is considered an appropriate model to assess mechanisms of diabetes and evaluate potential therapies.

Experimental design: Male Wistar-NIN rats with an average body weight of 120 gms are obtained from the National center for laboratory animal sciences, National institute of Nutrition, Hyderabad (NCLAS, NIN). Animals were maintained at NCLAS, MN and kept for acclimatization in an experimental room for two weeks. Diabetes was induced in overnight fasted animals by a single intraperitoneal injection of STZ (30 mg/kg) in 0.1 M citrate buffer, pH 4.5. Another set of rats, which received only a vehicle, served as the control (Group-C; n=6). Fasting blood glucose levels were measured 72 h after STZ injection. Animals having blood glucose levels >150 mg/dL were considered diabetic and all the animals are divided into four groups as shown in the Table 3 below.

TABLE 3

| | | No. of | |
|---|---|---|---|
| | Group | animals | Diet |
| I | Control | 6 | AIN 93 |
| II | Diabetic | 9 | AIN 93 |
| III | Diabetic + Soluble lutein (SL) | 8 | AIN 93 with |
| | | | soluble lutein 0.5% |
| IV | Diabetic + Regular lutein (RL) | 6 | AIN 93 with |
| | | | regular lutein 0.5% |

All the animals were housed in individual cages maintained on their respective diets for 12. weeks and drinking water was provided ad libitum throughout the study period. Daily food intake and weekly body weights, fasting glucose levels were noted. Before sacrifice electroretinogram was performed and glycosylated hemoglobin (HbAlC) was estimated. At the end of 12 weeks, rats were euthanized and retinas harvested for histological and molecular analysis (gene and protein expression).

Electroretinogram (ERG) Analysis: Diabetic retinoapthy is characterized by disturbances in retinal function. The retinal function can be assessed by electroretinogram. Diabetes results in ischemia and apoptosis in different retinal cell layers, which results in changes in the functions of the retina. It is well reported that oscillatory potentials (OPs, where OP represents waves, called oscillatory waves, which are a major component of ERG) are more affected in diabetes than a- or b-waves. OPs represent the functional aspects of inner retinal layers, ganglion cell layer and inner plexiform layer.

Animals were dark-adapted for overnight and prepared for the ERG procedure under dim red illumination. The pupils of the rats were dilated with atropine eye drops. The ground electrode was a subcutaneous needle in the tail, and the reference electrode was an ear clip electrode. The active contact lens electrodes were placed on the cornea. The recordings were performed with a UTAS Visual Diagnostic System. The responses were differentially amplified with a gain of 1,000 using alternating current-coupled UBA-4204 Amplifier. A flash stimuli of −2 to 8 dB were delivered via a with BigShot™ Ganzfeld System (LKC Technologies; Gaithersburg, Md., USA). The oscillatory potentials were extracted from the wave form and the sum of all OPs was calculated.

Quantitative real-time PCR: Total RNA was extracted from the retina of rats using Tri reagent. Isolated RNA was further purified by RNeasy Mini Kit (Qiagen) and quantified by measuring the absorbance at 260 and 280 nm on ND1000 spectrophotometer (NanoDrop technologies, Delaware, USA). The quality of RNA preparation was assessed by electrophoresis on a denaturing agarose gel. Two μg of total RNA was reverse transcribed using High Capacity cDNA Reverse Transcription kit. Reverse transcription reaction was carried out with a thermocycler (ABI 9700). Real-time polymerase chain reaction (PCR) (ABI-7500) was performed in triplicates with 25 ng cDNA templates using SYBR green master mix with gene specific primers. Normalization and validation of data were carried using (3-actin as an internal control and data were compared between samples according to comparative threshold cycle (2^{-ΔΔct}) method.

SDS-PAGE and Immunoblotting:Retina were homogenized in a buffer containing 20 mMTris, 100 mM NaCl, 1 mM ethylenediamine tetraacetic acid (EDTA) (TNE buffer; pH 7.5) containing 1 mM dithiothreitol (DTT), 1 mM phenylmethyl sulfonyl fluoride (PMSF), 1 μg/ml of each aprotinin, leupeptin, and pepstatin. The homogenate was centrifuged at 12,000×g for 20 min. The supernatant was collected and used for immunoblot analysis. Equal amount of protein from the supernatant was subjected to 12% SDS-PAGE and proteins were transferred onto polyvinylidene fluoride (PVDF) membrane. Nonspecific binding was blocked with 5% BLOT-QuickBlocker reagent (WB57, Calbiochem) in phosphate buffered saline with Tween (PBST) and incubated overnight at 4° C. with primary antibodies diluted in phosphate buffered saline (PBS). After washing with PBST, membranes were then incubated with anti-rabbit IgG (1:3500) secondary antibodies conjugated to horseradish peroxidase (HRP). The immunoblots were developed with enhanced chemiluminescence detection reagents (RPN2232, GE Health Care, Buckinghamshire, UK) and digital images were recorded by Image analyzer (Syngene, G-box). Quantification of band intensity was performed with Image J software.

Histopathology: The eye balls from selected animals were collected in fixative 4% paraformaldehyde solution in separately labeled vials. The tissues were given some nicks so as to facilitate penetration of fixative into deep tissue. They were kept at room temperature for 24-48 hrs followed by replacing the fixative with 20 mM sodium phosphate buffer. Buffer was scheduled to be replaced with fresh buffer one each week until histopathological processing. Tissues were embedded in paraffin and sections were taken in microtome. Coated slides are used for immunohistochemistry and immunofluorescence, whereas uncoated slides for hemotoxylin and eosin (H & E) staining.

Statistical Analysis: The data was expressed as mean±SEM. n=6; C (Non-diabetic control); D (Diabetic control); D+RL (diabetic+regular lutein); D+SL (Diabetic+soluble lutein); ***=p<0.001, **=P<0.01 and *=P<0.05 Vs C; ##=P<0.01 and #=P<0.05 Vs D.

Results were analyzed for statistical significance by one way ANOVA followed by Dunnett's multiple comparison test for comparing all the groups with control group. Between group significance was checked by two tailed unpaired t-test.

Results

Electroretinograph: In diabetic rats (D) amplitude of OPs were reduced (334.2 μV) compared to normal control (C) animals (498.4 μV). It is also noted that implicit time for OPs is increased in group-D. Ingestion of antioxidant lutein resulted in lowering the reduction in OP amplitudes, which is suggested by the sum of OPs; RL (442.6) and SL (561.9). Group-SL had shown significant difference in sum of OPs compared to group-D, in fact it is better than normal control rats.

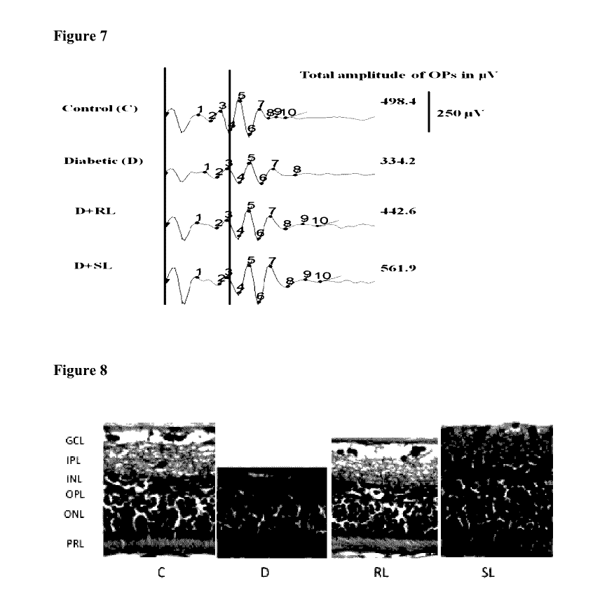

FIG. 7: Representative wave forms of OPs are shown from different groups and total amplitudes (FIG. 7 is shown in the drawings accompanied with the specification). C, Non diabetic control; D, Diabetic control; D+RL, Diabetes+Regular Lutein; D+SL, Diabetes+Soluble Lutein.

Morphology of retina: In control rats C, all the layers of the retina are intact and with maximum thickness of retina, and also noted with dense INL, and distinguishable separation (OPL) between INL and ONL. In contrast, diabetic retina (D) showed significantly reduced total retinal thickness and were also marked by less dense ONL and almost merged INL and ONLs. Treatment with lutein prevented gross morphological changes to a significant extent in diabetic retina. Soluble formulation of lutein was shown to be better than the regular lutein, indicated by dense ONLs (FIG. 8). FIG. 8 represents histology of retina, as shown in the drawings accompanied with the specification.

TABLE 4
Thickness of layers of retina (μ) of different groups.

| Retinal | Total retinal | | | | | |
|---|---|---|---|---|---|---|
| layers | thickness | GCL + IPL | INL | OPL | ONL | PRL |
| C | 121.74 | 46.813 | 10.767 | 5.024 | 25.697 | 19.426 |
| D | 58.848 | 12.508 | 9.257 | 0.791 | 23.052 | 12.184 |
| RL | 89.409 | 31.518 | 12.117 | 1.41 | 23.181 | 12.600 |
| SL | 112.714 | 40.877 | 16.074 | 4.718 | 31.196 | 23.072 |

A representative table of retinal layers thickness (in μm) measured by the use of Lieca application suit, Leica, Switzerland. C, Non diabetic control; D, Diabetic control; D+RL, Diabetes+Regular Lutein; D+SL, Diabetes+Soluble Lutein; GCL, ganglion cell layer; IPL, inner plexiform layer; INL, inner nuclear layer; OPL, outer plexiform layer; ONL, outer nuclear layer; PRL, photo receptor layer.

Expression of Retinal Markers (Genes) by Real-Time PCR, Immunohistochemistry and Immunoblotting:

Rhodopsin (Rho): Rhodopsin (Rho) is a biological pigment in photoreceptor cells of the retina that is responsible for the first events in the perception of light. The mRNA levels of Rho gene as quantified by real time PCR showed decreased levels in the retina of diabetic animals. Treatment with RL and SL prevented its decline and however feeding of SL had significant effect when compared to RC and even better than normal rats (FIG. 9A, as shown in the drawings accompanied with the specification). Further, we quantified protein levels of Rho by immunofluorescence, which is in coincidence with mRNA levels. Immunofluorescence imaging of Rho protein showed decreased expression in diabetic rat retina in comparison to normal control rat retina. Treatment with RL and SL prevented loss of Rho protein expression in diabetic retina indicated by intense Rho positive fluorescence. Furthermore, SL is found to be distinctly more effective than RL in preventing loss of Rho protein expression in rat retina in diabetic animals respectively (FIG. 9B, as shown in the drawings accompanied with the specification).

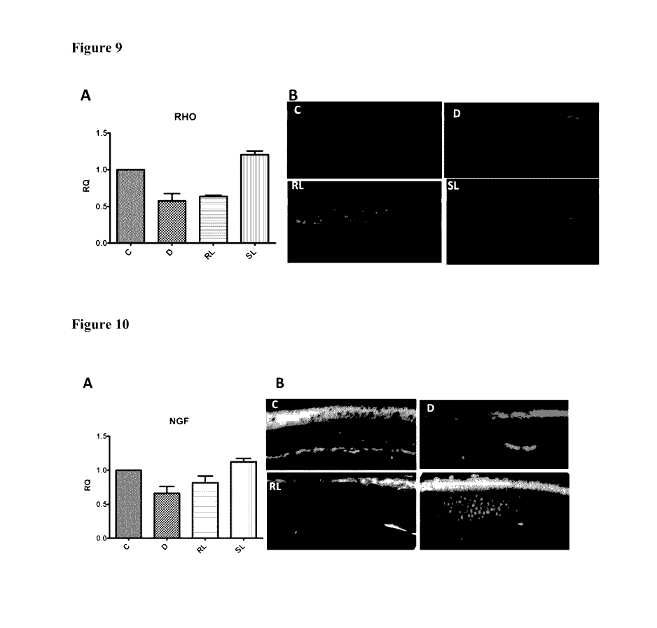

Nerve Growth Factor (NGF): NGF is the best-characterized neurotrophin, known to play a key role in the survival and differentiation of select neurons in the peripheral and central nervous system. Since its discovery in the 1950s, NGF has shown promise in the treatment of progressive neurodegenerative disorders. In animals, NGF is known to promote nerve terminal outgrowth and neuron recovery after ischemic, traumatic, and toxic injuries. We checked the status of NGF gene expression by real time PCR and found down regulation in the retinas of diabetic animals. Treatment with RL could not prevent its decline whereas SL prevented its down regulation (FIG. 10A, as shown in the drawings accompanied with the specification). The protein levels of NGF as estimated by immunofluorescence yielded similar results. Immunofluorescence imaging of NGF protein showed its decreased expression in diabetic rat retina in comparison to normal control rat retina (FIG. 10B, as shown in the drawings accompanied with the specification). Treatment with SL prevented loss of NGF protein in diabetic retina indicated by intense NGF positive fluorescence. RL was unable to prevent the loss of NGF protein effectively in diabetic rat retina.

Vascular endothelial growth factor (VEGF): Vascular endothelial growth factor (VEGF) is a signal protein produced by cells that stimulates vasculogenesis and angiogenesis. It is part of the system that restores the oxygen supply to tissues when blood circulation is inadequate. When VEGF is over expressed, it can contribute to disease. VEGF causes widespread retinal vascular dilation, produces breakdown of the blood-retinal barrier, and is implicated in ocular neovascularization. Western blotting for VEGF indicated the up regulation of VEGF expression in diabetic retina (FIG. 11, as shown in the drawings accompanied with the specification). Treatment with both RL and SL inhibited diabetes induced VEGF over expression.

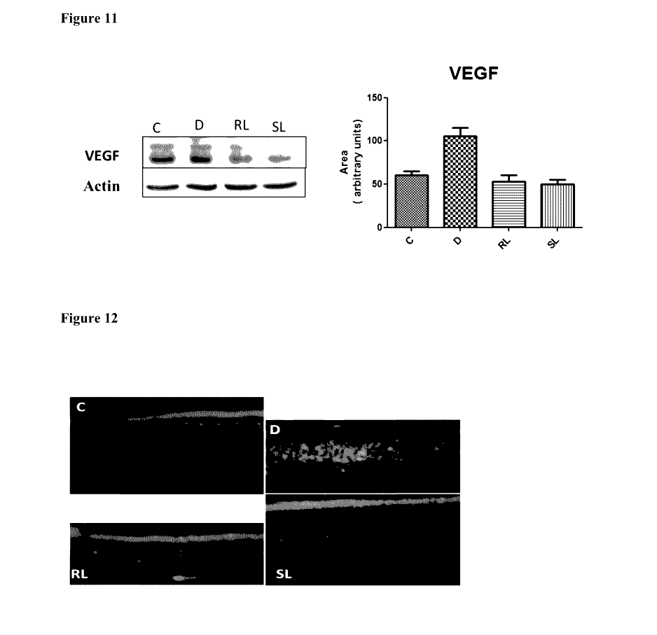

Platelet-derived growth factor (PDGF): PDGF is a growth factor that plays a significant role in blood vessel formation (angiogenesis), the growth of blood vessels from already-existing blood vessel tissue. Several studies have shown elevated PDGF concentrations in vitreous samples from patients with diabetic retinopathy. Like VEGF, PDGF is a proangiogenic growth factor that may promote aberrant neovascularization in diabetic retinopathy. Furthermore, PDGF may stimulate the formation and traction of epiretinal membranes in patients with diabetic retinopathy, leading to tractional retinal detachment. Indeed, the development of inhibitors that antagonize PDGF signaling in pathologic retinal neovascularization remains an active area of ophthalmic drug development. Immunohistochemistry for PDGF indicated the up regulation of protein in diabetic retina (FIG. 12, as shown in the drawings accompanied with the specification). Treatment with both RL and SL inhibited diabetes induced PDGF over expression.

Plasma lutein: To understand the effect of soluble lutein administration on its bioavailability plasma levels of carotenoids were measured by HPLC method. The data showed undetectable lutein levels in plasma of rats fed with normal diet (AIN-93). The plasma lutein levels were in detectable range in rats fed with AIN-93 diets mixed with regular lutein and soluble lutein. More interestingly, rats fed with SL diet were found to contain seven folds higher lutein levels compared with rats fed with RL diet. This in particular confirmed the success of this formulation in improving the bioavailability of lutein.

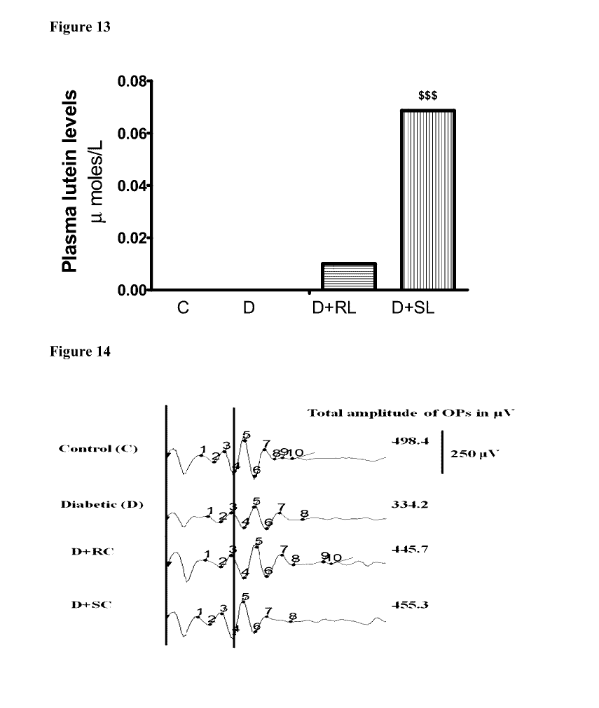

FIG. 13 represents serum lutein levels measured by reverse phase (RP)-HPLC, as shown in the drawings accompanied with the specification. Values are expressed as mean±SEM (n=6), $$$<0.001 D+SL Vs. D+RL.

Conclusion

Lutein administration to rats prevented diabetes induced abnormalities in the retina. Lutein retained the functionality of retina of rats which is lost in diabetic rats as checked by ERG. It is also evident by the morphological study of retina as done by H & E staining. Lutein prevented decline in the expression of rhodopsin and nerve growth factor (NGF) which have vital roles in maintaining health of the retina. Lutein prevented over expression of VEGF and PDGF that are involved in stress and angiogenesis. Interestingly rats treated with soluble lutein showed profound benefit when compared with regular lutein, and the increased bioavailability is shown by the increased plasma levels. Further, the antioxidant and anti-inflammatory potential of lutein may contribute for its beneficial effect. Hence soluble lutein can be used to treat and or prevent diabetic retinopathy.

### Example 3

### Effect of UltraSol CurcuWin™ in Diabetic Retinopathy by Nutrigenomics Approach

The effect of soluble curcumin (UltraSol CurcuWin™) was investigated in diabetic rats with respect to its beneficial effect on retina by nutrigenomics approach and the effect was compared with regular curcumin.

Methodology

Same as mentioned in example 2. All the animals were divided into four groups as shown below

TABLE 5

| | | No. of | |
|---|---|---|---|
| | Group | animals | Diet |
| I | Control | 6 | AIN 93 |
| II | Diabetic | 9 | AIN 93 |
| III | Diabetic + Soluble lutein (SC) | 8 | AIN 93 with soluble |
| | | | curcumin 0.01% |
| IV | Diabetic + Regular lutein (RC) | 6 | AIN 93 with regular |
| | | | curcumin 0.01% |

Electroretinograph: In diabetic rats (D) amplitude of OPs were reduced (334.2 μV) compared to normal control (C) animals. It is also noted that implicit time for OPs is increased in group-D. Ingestion of antioxidant curcumin resulted in lowering the reduction in OP amplitudes suggested by the sum of OPs; RC (445.7), SC (455.3). SC not only lowered the reduction in sum of OPs, but also normalized the implicit times.

FIG. 14 represents wave forms of OPs from individual animals of different groups and total amplitudes, as shown in the drawings accompanied with the specification. C, Non diabetic control; D, Diabetic control; D+RC, Diabetes+Regular Curcumin; D+SC, Diabetes+Soluble Curcumin.

Morphology of Retina: In control rats C, all the layers of the retina are intact and with maximum thickness of retina and also noted with dense INL, and distinguishable separation (OPL) between INL and ONL. In contrast, diabetic rats retina (D) showed significantly reduced total retinal thickness and were also marked by less dense ONL and almost merged INL and ONLs. Treatment with curcumin prevented gross morphological changes to a significant extent in diabetic retina. Soluble formulation of this active principle was shown to be better than the regular active principle, indicated by dense ONLs.

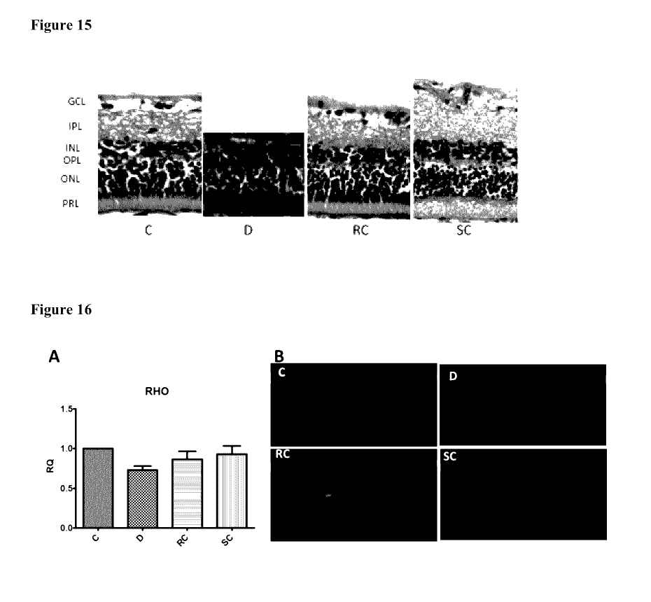

FIG. 15, Representative histology of retina, as shown in the drawings accompanied with the specification.

TABLE 6
Thickness of layers of retina (μ) of different groups.

| Retinal | Total retinal | | | | | |
|---|---|---|---|---|---|---|
| layers | thickness | GCL + IPL | INL | OPL | ONL | PRL |
| C | 121.74 | 46.813 | 10.767 | 5.024 | 25.697 | 19.426 |
| D | 58.848 | 12.508 | 9.257 | 0.791 | 23.052 | 12.184 |
| RC | 106.014 | 35.692 | 13.648 | 5.685 | 30.108 | 19.520 |
| SC | 124.430 | 54.004 | 14.926 | 6.597 | 29.320 | 21.857 |

A representative table of retinal layers thickness (in μm) measured by the use of Lieca application suit, Leica, Switzerland. C, Non diabetic control; D, Diabetic control; D+RC, Diabetes+Regular Curcumin; D+SC, Diabetes+Soluble Curcumin. GCL, ganglion cell layer; IPL, inner plexiform layer; INL, inner nuclear layer; OPL, outer plexiform layer; ONL, outer nuclear layer; PRL, photo receptor layer.

Expression of Retinal Markers (Genes) by Real-Time PCR, Immunohistochemistry and Immunoblotting:

Rhodopsin (Rho): Rhodopsin, is a biological pigment in photoreceptor cells of the retina that is responsible for the first events in the perception of light. The mRNA levels of Rho gene as quantified by real time PCR showed decreased levels in the retina of diabetic animals. Treatment with RC and SC prevented its decline and however SC treatment has more beneficiary effect when compared to RC (FIG. 16.A, as shown in the drawings accompanied with the specification). Protein levels of Rho were quantified by immunofluorescence, which is in coincidence with mRNA levels. Immunofluorescence imaging of Rho protein showed its decreased expression in diabetic rat retina in comparison to normal control rat retina. Treatment with RC and SC prevented loss of Rho protein expression in diabetic retina indicated by intense Rho positive fluorescence. Furthermore, SC is found to be more effective than RC in preventing loss of Rho protein expression in rat retina in diabetic animals respectively (FIG. 16.B, as shown in the drawings accompanied with the specification).

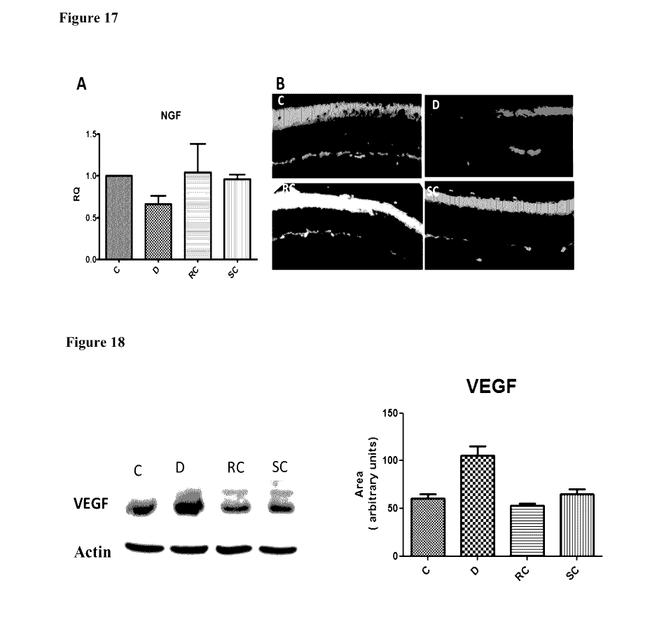

Nerve Growth Factor (NGF): NGF is the best-characterized neurotrophin, known to play a key role in the survival and differentiation of select neurons in the peripheral and central nervous system. Since its discovery in the 1950s, NGF has shown promise in the treatment of progressive neurodegenerative disorders. In animals, NGF is known to promote nerve terminal outgrowth and neuron recovery after ischemic, traumatic, and toxic injuries. We checked the status of NGF gene expression by real time PCR and found down regulation in the retinas of diabetic animals. Treatment with both regular as well as soluble curcumin showed equal beneficiary effect in preventing down regulation of NGF gene under diabetic conditions (FIG. 17.A, as shown in the drawings accompanied with the specification). The protein levels of NGF as estimated by immunofluorescence yielded similar results. Immunofluorescence imaging of NGF protein showed its decreased expression in diabetic rat retina in comparison to normal control rat retina (FIG. 17.B, as shown in the drawings accompanied with the specification). Treatment with RC and SC prevented loss of NGF protein in diabetic retina indicated by intense NGF positive fluorescence.

Vascular Endothelial Growth Factor (VEGF):

Vascular endothelial growth factor (VEGF) is a signal protein produced by cells that stimulates vasculogenesis and angiogenesis. It is part of the system that restores the oxygen supply to tissues when blood circulation is inadequate. When VEGF is over expressed, it can contribute to disease. VEGF causes widespread retinal vascular dilation, produces breakdown of the blood-retinal barrier, and is implicated in ocular neovascularization.

Western blotting for VEGF indicated the up regulation of VEGF expression in diabetic retina (FIG. 18, as shown in the drawings accompanied with the specification). Treatment with both RL and SL inhibited diabetes induced VEGF over expression.

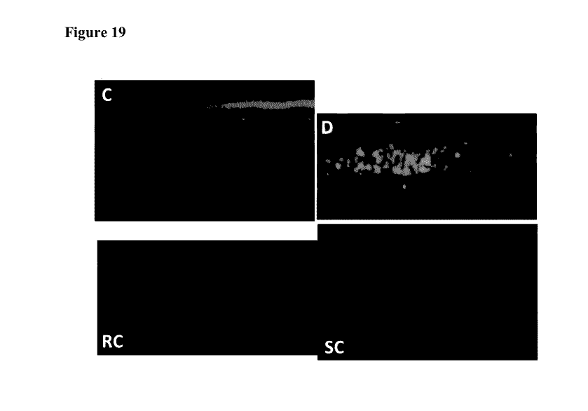

Platelet-derived growth factor (PDGF): PDGF is a growth factor that regulates cell growth and division. In particular, it plays a significant role in blood vessel formation (angiogenesis), the growth of blood vessels from already-existing blood vessel tissue. Several studies have shown elevated PDGF concentrations in vitreous samples from patients with diabetic retinopathy. Like VEGF, PDGF is a proangiogenic growth factor that may promote aberrant neovascularization in diabetic retinopathy. Furthermore, PDGF may stimulate the formation and traction of epiretinal membranes in patients with diabetic retinopathy, leading to tractional retinal detachment. Indeed, the development of inhibitors that antagonize PDGF signaling in pathologic retinal neovascularization remains an active area of ophthalmic drug development. Immunohistochemistry for PDGF indicated the up regulation of protein in diabetic retina (FIG. 19, as shown in the drawings accompanied with the specification). Treatment with both RC and SC inhibited diabetes induced PDGF over expression. Soluble curcumin was more effective than regular curcumin (FIG. 19, as shown in the drawings accompanied with the specification).

Conclusion

Curcumin administration to rats prevented diabetes induced abnormalities in the retina. Curcumin retained the functionality of retina of rats which is lost in diabetic rats as checked by ERG. It is also evident by the morphological study of retina as evaluated by H & E staining where thickness of various layers of retina was measured. Curcumin prevented decline in the expression of rhodopsin and nerve growth factor which have vital role. Curcumin prevented over expression of VEGF and PDGF that are involved in stress and angiogenesis. Interestingly rats treated with soluble curcumin showed profound benefit when compared with regular curcumin and this might be due to increased bioavailability. Hence soluble curcumin can be used to treat and or prevent diabetic retinopathy.

### Advantages

1. Provides dry free flowing, water soluble/miscible form of lipophilic nutrient.
2. Provides a suitable method of delivering poorly water soluble, oily, lipophilic nutrient in the form of granule, powder, tablets, ointment, paste, mouth wash, gargle, sachet, capsules or in to beverages.
3. Provides a dosage form of poorly water soluble, oily, lipophilic nutrient with high bioavailability.
4. The compositions herein are effective to regulate blood glucose levels.
5. The compositions and methods herein containing lipophilic nutrients are effective to regulate amacrine cells dysfunction in diabetic retinopathy.
6. The compositions herein are effective to prevent loss of retinal layers, Rhodopsin levels and NGF protein levels in diabetic retinopathy.
7. The compositions herein are effective to inhibit diabetes induced PDGF over expression in diabetic retinopathy.
8. The compositions herein are effective to prevent accumulation of in-soluble lens proteins in diabetic cataract.
9. The compositions herein are effective to prevent accumulation of sorbitol levels in lens in diabetic cataract.
10. The compositions herein are effective to reduce protein aggregation and normalize the profile of total soluble protein in diabetic cataract.

1. A method for delaying the development and maturation of eye related complications of diabetes, comprising administering to an animal a composition which is safe for human consumption and useful as a dietary supplement for nutrition and health promoting benefits, wherein the composition is a molecular dispersion comprising a lipophilic nutrient, a stabilizer, and a water-soluble hydrophilic carrier, and wherein the composition is administered in an amount effective to delay eye related complications of diabetes.

2. The method of claim 1, wherein the eye related complications of diabetes is cataract and retinopathy.

3. The method as claimed in claim 1, wherein the lipophilic nutrient is selected from a group comprising lutein, lutein isomers, lutein ester, zeaxanthin isomers, turmeric extract, curcumin, ginger, and the mixtures thereof.

4. The method as claimed in claim 1 any of the presiding claims, wherein the step of administering includes administering the composition in an effective amount in regulating blood glucose levels.

5. The method as claimed in claim 1, wherein said step of administering includes administering the composition in an effective amount in regulating glycated hemoglobin (HbAlc) levels.

6. The method as claimed in claim 1, wherein said step of administering includes administering the composition in an effective amount in regulating amacrine cells dysfunction in diabetic retinopathy.

7. The method as claimed in claim 1, wherein the step of administering includes administering the composition in an effective amount in preventing loss in retinal layers, Rhodopsin levels and NGF protein levels in diabetic retinopathy.

8. The method as claimed in claim 1, wherein the step of administering includes administering the composition in an effective amount in inhibiting diabetes induced PDGF over expression in diabetic retinopathy.

9. The method as claimed in claim 1, wherein the step of administering includes administering the composition in an effective amount in preventing accumulation of in-soluble lens proteins in diabetic cataract.

10. The method as claimed in claim 1, wherein the step of administering includes administering the composition in an effective amount in preventing accumulation of sorbitol levels in lens in diabetic cataract.

11. The method as claimed in claim 1, wherein the step of administering includes administering the composition in an effective amount in reducing protein aggregation and in normalizing the profile of total soluble protein in diabetic cataract.

12. The method as claimed in claim 1, wherein the composition comprises a surfactant.

13. The method as claimed in claim 1, wherein the composition contains at least 80% by weight of total xanthophylls, out of which the trans-lutein content is 80-95% w/w; (R,R)-zeaxanthin is 14-20% w/w; (R,S)-zeaxanthin is 0.01-1% w/w; or contains translutein content is 80-95% w/w; (R,R)-zeaxanthin is 14-20% w/w, and traces of other carotenoids derived from the plant extracts/oleoresin containing xanthophylls/xanthophylls esters; or contains curcumin which contains 5-95% of curcuminoids.

14. The method as claimed in claim 1, wherein the stabilizer is selected from Ascorbic acid, BHA, BHT, ascorbyl palmitate, rosemary extract, mixed natural tocopherols, alpha tocopheryl acetate, sodium ascorbate, castor oil derivatives, sodium lauryl sulfate and mixtures thereof.

15. The method as claimed in claim 1, wherein the water-soluble hydrophilic carrier used is selected from polyethylene glycol 200, polyethylene glycol 400, ethylene glycol, propylene glycol, glycerol, sorbitol, glucose syrup, corn steep liquor, mannitol, polyethylene glycol 6000, polyethylene glycol 10000, Polyethylene glycol 20000, polyvinyl pyrrolidone, hydroxyl propyl methyl cellulose, sucrose, glucose, sodium chloride, hydroxyl propyl cellulose, polyvinyl alcohol, soluble starch, hydrolyzed starch and mixtures thereof.

16. The method as claimed claim 12, wherein the surfactant is selected from a group comprising polysorbate 20, polysorbate 60, polysorbate 80, lecithin, sucrose fatty acid esters, glyceryl fatty acid esters, sodium lauryl sulfate and mixtures thereof.

17. The method as claimed in claim 1, wherein the composition is in the form of powders, tablets, capsules, sachets, beadlets, microencapsulated powders, oil suspensions, liquid dispersions, pellets, soft gel capsules, chewable tablets or liquid preparations.
